(19) United States
(12) Patent Application Publication
(10) Pub. No.: US 2022/0079507 A1
(43) Pub. Date: Mar. 17, 2022

(54) SYSTEM AND METHOD TO MEASURE AND MONITOR NEURODEGENERATION

(71) Applicants: ICM (INSTITUT DU CERVEAU ET DE LA MOELLE ÉPINIÈRE), Paris (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITÉ, Paris (FR)

(72) Inventors: Stephane EPELBAUM, Paris (FR); Sinead GAUBERT, Paris (FR); Federico RAIMONDO, Embourg (BE); Jacobo D SITT, Paris (FR); Lionel NACCACHE, Paris (FR)

(73) Assignees: ICM (INSTITUT DU CERVEAU ET DE LA MOELLE ÉPINIÈRE), Paris (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITÉ, Paris (FR)

(21) Appl. No.: 17/299,898

(22) PCT Filed: Dec. 20, 2019
(86) PCT No.: PCT/EP2019/086629

§ 371 Date: Jun. 4, 2021

(30) Foreign Application Priority Data
Dec. 21, 2018 (EP) 18306817.0

(51) Int. Cl. A61B 5/00; A61B 5/374

(52) U.S. Cl. CPC A61B 5/4088; A61B 5/374

(57) ABSTRACT

A system to measure and monitor neurodegeneration of a subject, which includes: an acquisition module configured to acquire electroencephalographic signals with multiple EEG channels from a subject perceptually isolated; a calculation module configured to extract at least one EEG metric representative of neurodegeneration; and an evaluation module configured to evaluate the at least one EEG metric and extract a neurodegeneration index.

### FIELD OF INVENTION

The present invention pertains to the field of measuring and monitoring of neurodegeneration by assessment of changes in neuromarkers. In particular, the invention relates to the monitoring of alterations of specific neuromarkers in preclinical Alzheimer disease subjects using electroencephalogram measurements.

### BACKGROUND OF INVENTION

Alzheimer's disease (AD) is the most common form of dementia, as it accounts for an estimated 60 to 80 percent of cases. The pathophysiological process of Alzheimer's disease begins many years before the onset of symptoms. It is essential to diagnose Alzheimer's disease as early as possible because patients will be more likely to benefit from disease modifying treatments if treated early in the disease course, before major brain damage has occurred. It is therefore important to develop neuromarkers that are sensitive to this early, “preclinical” stage of Alzheimer's disease even before mild cognitive impairment (MCI) occurs. At the preclinical stage subjects are cognitively unimpaired but show evidence of cortical amyloid-β (Aβ) deposition which is considered to be the most upstream process in the pathological cascade of Alzheimer's disease and is measured by amyloid PET or decreased amyloid-β₁₋₄₂ and amyloid-β₁₋₄₂/amyloid-β₁₋₄₀ ratio in the cerebrospinal fluid (CSF). Aβ deposition can be associated to pathologic tau deposits, measured by tau PET or elevated CSF phosphorylated tau and to neurodegeneration that is revealed by elevated CSF total tau, ¹⁸F-fluorodeoxyglucose (¹⁸F-FDG) PET hypometabolism in an Alzheimer's disease-like pattern and atrophy on MRI. However, those imaging techniques are not easily available and are expensive in terms of purchasing equipment.

Neuromarkers for Alzheimer's disease are important not only for identifying individuals at high risk of preclinical Alzheimer's disease, but also to better understand the pathophysiological processes of disease progression.

In this context, EEG represents an interesting alternative due to its numerous advantages as it is a non-invasive, cheap and a reproducible technique, that directly measures neural activity with a good temporal resolution.

There is already a rich literature on the use of EEG neuromarkers in mild cognitive impairment and Alzheimer's disease, such as spectral measures and synchronization between brain regions. Patients with Alzheimer's disease or MCI usually show slowing of oscillatory brain activity, reduced EEG complexity and reduced synchrony. Decreased alpha power correlated with hippocampal atrophy and lower cognitive status. Growing evidence show that Alzheimer's disease targets cortical neuronal networks related to cognitive functions, which is revealed by the impairment in functional connectivity in long range networks. There are several types of measures of functional connectivity using EEG or magnetoencephalography (MEG) including spectral coherence, synchronization likelihood or information theory indexes. A decrease of alpha coherence, an increase of delta total coherence and an abnormal alpha fronto-parietal coupling have been described in AD. A reduction of alpha and beta synchronization likelihood was shown in MCI and AD. An EEG study in older people with subjective memory complaints found no association between cortical amyloid load and, whereas another study using MEG in cognitively normal individuals at risk for Alzheimer's disease showed altered FC in the default mode network (DMN). However, the usefulness of EEG characteristics as neuromarkers for the evaluation of preclinical Alzheimer's disease is not yet established, as most studies have focused on EEG neuromarkers at later stages of the disease, after the onset of symptoms.

The present invention proposes a system and a method using neuromarkers sensitive to the preclinical stage of Alzheimer's disease in order to measure and monitor neurodegeneration in a subject.

### SUMMARY

A first aspect of the present invention relates to a system to measure and monitor neurodegeneration of a subject, comprising:
an acquisition module configured to acquire electroencephalographic signals with multiple EEG channels from a subject perceptually isolated;
a calculation module configured to extract at least one EEG metric representative of neurodegeneration; and
an evaluation module configured to evaluate the at least one EEG metric and extract a neurodegeneration index.

According to one embodiment, the neurodegeneration index is representative of the neurodegeneration affecting a subject suffering from preclinical Alzheimer's disease.

According to one embodiment, the neurodegeneration index is representative of the stage of preclinical Alzheimer's disease affecting the subject.

The present invention provides a system configured to extract a reliable neurodegeneration index using at least one neuromarker sensitive to early “preclinical” stage of Alzheimer's disease even before mild cognitive impairment (MCI) occurs. This aspect is of great interest since the detection in a subject of preclinical stage of Alzheimer's disease will have a major impact on the treatment of Alzheimer's disease. Indeed, an early intervention may offer the best chance of therapeutic success.

According to one embodiment, the acquisition module comprises at least two EEG channels.

According to one embodiment, the acquisition module comprises at least four EEG channels, for example two channels place on the frontal area and two channels placed on the parietal area. Advantageously the use of a low number of electrodes allows to acquire a lower volume of raw data that may be rapidly analyzed so as to obtain the neurodegeneration index almost in real time. Furthermore, an acquisition module having fewer electrode is of easier conception or easier accessibility.

According to one embodiment, the calculation module is configured to extract at least one EEG metric selected from the group of: weighted symbolic mutual information in at least one frequency band, power spectral density calculated in at least one frequency band, median spectral frequency, spectral entropy and/or algorithmic complexity.

According to one embodiment, in order to extract the weighted symbolic mutual information, the calculation module is configured to perform a symbolic transformation of the electroencephalographic signals into a series of discreate symbols and calculating the weighted symbolic mutual information using said series of discrete symbols.

According to one embodiment, the weighted symbolic mutual information is calculated in the theta frequency band.

The dominant resting state rhythms are typically observed at theta frequencies and this rhythm shows maximum changes in Alzheimer's disease patients. Therefore, the weighted symbolic mutual information in the theta frequency band advantageously contains information allowing the discrimination between non-preclinical Alzheimer's disease subjects and Alzheimer's disease subjects.

According to one embodiment, the power spectral density is calculated in the delta frequency band, theta frequency band, alpha frequency band, beta frequency band and/or in the gamma frequency band.

According to one embodiment, the EEG metrics extracted by the calculation module further comprises at least one of the following median spectral frequency, spectral entropy or algorithmic complexity.

According to one embodiment, the evaluation module is configured to extract the neurodegeneration index from the comparison of the at least one EEG metrics with at least one predefined threshold.

According to one embodiment, the system further comprises a pre-processing module to preprocess the electroencephalographic signals.

According to one embodiment, the system further comprises a user interface module providing the neurodegeneration index as output.

A second aspect of the present invention relates to a computer-implemented method for measuring and monitoring neurodegeneration of a subject, comprising the steps of:
receiving electroencephalographic signals acquired with multiple EEG channels from a subject perceptually isolated;
extracting at least one EEG metric representative of neurodegeneration;
evaluating the at least one EEG metric and extracting a neurodegeneration index; and
outputting the neurodegeneration index.

According to one embodiment, the at least one EEG metric, extracted at the extraction step of the computer-implemented method, is selected from the group of: weighted symbolic mutual information in at least one frequency band, power spectral density calculated in at least one frequency band, median spectral frequency, spectral entropy and/or algorithmic complexity.

One of the main strengths of the present system and method is the implementation of a high-performing and practical EEG processing pipeline with automated artefact elimination and extraction of several validated EEG neuromarkers (i.e. EEG metrics). This tool avoids the need for the time-consuming manual removal of artefacts and the risk of possible human biases.

The system and method of the present invention present the great advantage of using electroencephalogram, which is a non-invasive, cheap and widely-available technique, and therefore could be used as a screening tool for identifying individuals at high risk of neurodegeneration and future cognitive decline.

Another aspect of the present invention relates to a computer program comprising instructions, which when the program is executed by a computer, causes the computer to carry out the steps of the method according to any one of the embodiments described here above.

Yet another aspect of the present invention relates to a computer-readable storage medium comprising instructions that when executed by a computer, causes the computer to carry out the steps of the method according to any one of the embodiments described here above.

Definitions

In the present invention, the following terms have the following meanings:
“Alzheimer's disease” is defined by the positivity of neuromarkers of both amyloidopathy (A1) and tauopathy (T1) in line with the pathologic definition of the disease.
“Clinical Alzheimer's disease” refers to a clinical stage of the Alzheimer's disease defined by the occurrence of the clinical phenotype of Alzheimer's disease (either typical or atypical) and which encompasses both the prodromal and the dementia stages.
“Preclinical Alzheimer's disease” refers to a preclinical stage before the onset of the clinical phenotype.
“Epoch” refers to a determined period of the electroencephalographic signal that is analyzed independently. Epochs are not overlapping.
“Electroencephalogram” refers to the record of the electrical activity of the brain of a subject.
“Electrode” refers to a conductor used to establish electrical contact with a nonmetallic part of a circuit, preferably a subject body. For instance, EEG electrodes are small metal discs usually made of stainless steel, tin, gold, silver covered with a silver chloride coating; there are placed on the scalp at specific positions.
“Subject” refers to a mammal, preferably a human.
“Mini-Mental State Examination score” or “MMSE” refers to a 30-point questionnaire that is used extensively in clinical and research settings to measure cognitive impairment.
“RL/RI-16 test” refers to the French adaptation of the “Free and Cued Selective Reminding Test” configured to evaluate the presence and nature of verbal episodic memory difficulties so as to detect worsening or progression to dementia in individuals with mild cognitive deficits.
“Frontal Assessment Battery or FAB” refers to a neurophysiological test developed by Dubois and Pillon in 2000 to determine and evaluate frontal lobe disorder.

### BRIEF DESCRIPTION OF THE DRAWINGS

FIG. 1 shows a block diagram representing the steps implemented by the method of the present invention according to a first embodiment.

FIG. 2 shows for one subject 256 electrodes topographical maps of the most discriminatory EEG metrics. The topographical 2D projection (top=front) of each measure [normalized power spectral density in delta (PSD deltaₙ), beta (PSD betaₙ), gamma (PSD gammaₙ), median spectral frequency (MSF), spectral entropy (SE), algorithmic complexity (K) and weighted symbolic mutual information in theta band (wSMI θ)] is plotted for preclinical Alzheimer's disease group and control group (columns). The third column indicates whether the two groups were significantly different from one another, using a linear mixed model (black=P<0.01, scale of grey=P<0.05, white=not significant; all p-values are adjusted on gender, amyloid SUVR and ApoE4 status). The fourth column indicates the multiple comparison corrected p-values on 10 measures according to the Benjamini-Hochberg procedure. P-values for main effect are displayed if there was no significant interaction between electrode and main effect. In case of significant main effect and significant interaction, p-values of post hoc tests at electrode level are shown.

FIG. 3 shows average measures of EEG metrics across all electrodes for control group and preclinical Alzheimer's disease group. Estimated marginal means and standard deviation are depicted; significant adjusted p-values on age, gender, education, amyloid SUVR and ApoE4 status are indicated with *P<0.05, **P<0.01, n.s. not significant; boxed metrics have a BH FDR-corrected p-value<0.05.

FIGS. 4A and 4B shows local regression of average measures of EEG metrics across all electrodes as a function of ¹⁸F-florbetapir PET SUVR values (MSF=median spectral frequency; PSD=power spectral density; SE=spectral entropy; wSMI=weighted symbolic mutual information).

FIG. 5 shows linear and least squares regression of average EEG metrics as a function of ¹⁸F-florbetapir SUVR to determine amyloid PET SUVR inflection points. The results are only shown for EEG metrics with a p-value<0.05. P-values are adjusted on group, gender and ApoE4 status and are corrected for multiple comparison testing by the Benjamini-Hochberg procedure. (MSF=median spectral frequency; PSD=power spectral density; SE=spectral entropy).

FIG. 6 shows comparison of inter-cluster functional connectivity matrices between preclinical Alzheimer's disease and control group. The third matrix indicates whether the two groups were significantly different from one another, using a linear mixed model (black=P<0.01, scale of grey=P<0.05, white=not significant; all p-values are adjusted on gender, amyloid SUVR and ApoE4 status). wSMI=weighted symbolic mutual information.

FIG. 7 shows local regression of average EEG metrics across all scalp electrodes as a function of amyloid SUVR (SE=spectral entropy).

FIG. 8 shows local regression of average EEG metrics across all scalp electrodes as a function of amyloid SUVR for neurodegeneration positive subjects only (SE=spectral entropy).

FIG. 9 shows Local regression of average EEG metrics across all scalp electrodes as a function of mean FDG SUVR (FDG=fluorodeoxyglucose; SE=spectral entropy).

FIGS. 10A and 10B shows a 224 electrodes topographical maps of EEG metrics. The topographical 2D projection (top=front) of each measure [normalized power spectral density in delta (δ), theta (θ), alpha (α), beta (β), gamma (λ), median spectral frequency (MSF), spectral entropy (SE), algorithmic complexity (K) and weighted symbolic mutual information in theta band and alpha band (wSMI θ and wSMI α)] is plotted for the A+N+ group, the A−N+ group, A+N− group and control group A−N− (columns) Statistics were done on 224 electrodes by non-parametric cluster permutation test. The three last columns indicate non-parametric cluster-based permutation test results for the pairwise comparisons: A+N+ versus A−N−; A−N+ versus A−N−; and A+N− versus A−N− for each EEG metric. The topographical maps in the three last columns are color-coded according to the cluster permutation tests P-values (color: P50.05, greyscale: P40.05). Clusters of electrodes whose EEG metrics' values are significantly different from the control group (A−N−) are depicted.

FIG. 11 shows an evaluation of the performance of three classifiers (decision tree, logistic regression and Random forest) with different isolated variables combined to classify the N+ and N− subjects. The distribution of the AUC values is represented with the median and the IC95%. DEMO_sansAPOE=demography (age, sex, education level); DEMO_avecAPOE=demography (age, sex, education level) plus ApoE4 status, PSY=neurophysiological score (MMSE, RL/RI-16, FAB); EEG=10 EEG metrics averaged on 224 electrodes; HV=hippocampus volume.

FIG. 12 shows the evolution of the detection of the status N+ versus N− as a function of the reduction of the number of the EEG electrodes (224, 128, 64, 32, 16, 8, 4, 2). The good classification rate, sensitivity and specificity obtained with logistic regression are indicated with the median and 95% CI to maximize the Youden index (sensitivity+specificity−1).

### DETAILED DESCRIPTION

The following detailed description will be better understood when read in conjunction with the drawings. For the purpose of illustrating, the method is shown in the preferred embodiments. It should be understood, however that the application is not limited to the precise arrangements, structures, features, embodiments, and aspect shown.

The present invention relates to a system and a method configured to measure and monitor neurodegeneration in a subject by extracting resting state EEG neuromarkers of neurodegeneration associated to high risk of preclinical AD.

One aspect of the present invention concerns a method comprising multiple step configured to measure and monitor neurodegeneration of a subject.

According to one embodiment, said method is a computer-implemented method.

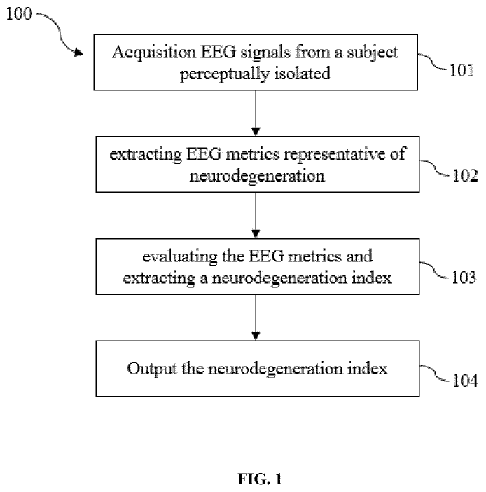

According to the embodiment show in FIG. 1, the first step 101 of the method 100 consists in the reception of at least two electroencephalographic signals of a subject. Said electroencephalographic signals being acquired with an electroencephalogram system having at least two electrodes, positioned onto predetermined areas of the scalp of the subject in order to obtain a multi-channel electroencephalographic signal. According to one embodiment, the electroencephalographic signals are acquired by at least 2, 4, 8, 10, 15, 16, 17, 18, 19, 20, 21, 32, 64, 128 or 256 electrodes. The details concerning the type of electroencephalogram system from which the EEG signals are acquired are provided in the embodiments below concerning the system of the present invention.

As a variant, the first step may consist in the transmission of instruction to an electroencephalogram system in order to control the acquisition of multiple EEG signals from the subject and receive said signals in real time. The electroencephalographic signals may be alternatively received from a medical database where the EEG signals may have been previously stored.

According to one embodiment, the electroencephalographic signals received are acquired on a subject that is placed in a condition of perceptual isolation, meaning that stimuli to one or more of the senses of the subject are deliberately reduced or removed.

According to one preferred embodiment, the EEG signals acquisition is performed on a subject positioned in a quiet room and instructed to maintain his eyes closed during the whole acquisition. This has the advantage of facilitating the extraction of resting state EEG neuromarkers of neurodegeneration.

According to one embodiment, the method comprises a pre-processing step for pre-processing of the electroencephalographic signals in order to remove or reject noise. According to one embodiment, the electroencephalographic signals are further pre-processed in order to remove or reject artefact.

According to one embodiment, the electroencephalographic signals from individual electrodes are digitally filtered with at least one filter chosen from group: low-frequency reject filter, high-frequency reject filter, bandpass filter, band stop filter. In one example, electroencephalographic signals may be filtered using first-order Butterworth band-pass filter and a third-order Butterworth notch filter; a skilled artisan would be able to select a suitable range of frequencies to reject.

According to one embodiment, the pre-processing step is further configured to divide the prerecorded electroencephalographic signal into non-overlapping consecutive segments of fixed length also called epochs. According to one embodiment, said fixed length of segments is of the order of the second, for example 0.5, 1, 2, or 3.

One or more of the following frequency bands may be extracted during the filtering process: delta band (typically from about 1 Hz to about 4 Hz), theta band (typically from about 3 to about 8 Hz), alpha band (typically from about 7 to about 13 Hz), low beta band (typically from about 12 to about 18 Hz), beta band (typically from about 17 to about 23 Hz), and high beta band (typically from about 22 to about 30 Hz). Higher frequency bands, such as, but not limited to, gamma band (typically from about 30 to about 80 Hz), are also contemplated.

According to one embodiment, the artefacts are corrected from the electroencephalographic signal using one or a combination of the following techniques: adaptive filtering, Wiener filtering and Bayes filtering, Hilbert-Huang Transform filter regression, blind source separation (BSS), wavelet transform method, empirical mode decomposition, nonlinear mode decomposition and the like.

One of the main sources of physiological noise arises from eye movements and more precisely from eye blinks which generates large amplitude signals in the electroencephalographic signals. Those ocular artefacts present a wide spectral distribution thus perturbing all classic electroencephalographic bands, including the alpha band which is the band of interest in the method disclosed by the present invention.

In a one embodiment, the ocular artefacts are corrected using blind source separation (BSS) or regression on an electrooculogram trace.

According to one embodiment, the method 100 of the present invention comprises a calculation step 102 configured to extract at least one EEG metric representative of the neurodegeneration in a subject.

According to one embodiment, the neurodegeneration index extracted at the calculation step is representative of the neurodegeneration.

According to one embodiment, the neurodegeneration index extracted at the calculation step is representative of the neurodegeneration corresponds to suspected non-Alzheimer's disease pathophysiology.

According to one embodiment, the neurodegeneration index extracted at the calculation step is representative of the neurodegeneration affecting a subject suffering from preclinical Alzheimer's disease.

The at least one EEG metric may be selected from the following group: weighted symbolic mutual information in at least one frequency band, power spectral density calculated in at least one frequency band, median spectral frequency, spectral entropy and/or algorithmic complexity.

The weighted symbolic mutual information (wSMI) is an information-theoretic metric that is used to quantify global information sharing, which evaluates the extent to which two EEG signals present non-random joint fluctuations, suggesting that they share information.

According to one embodiment, the extraction of the weighted symbolic mutual information is preceded by a step consisting in performing a symbolic transformation or an equivalent mathematical mapping of the electroencephalographic signals into a series of discreate symbols.

The symbolic transformation depends on the length of the symbols and their temporal separation. The symbolic transformation may be performed by first extracting sub-vectors of the EEG signal recorded from a given electrode, each comprising n epochs separated by a fixed temporal separation. The temporal separation thus determines the broad frequency range to which the symbolic transform is sensitive. Each sub-vector is then assigned to a unique symbol, depending only on the order of its amplitudes. For a given symbol length (n), there are n! possible orderings and thus equal number of possible symbols. In EEG signals, symbols may not be equiprobable, and their distribution may not be random either over time or over the different sensor locations. The weighted symbolic mutual information evaluates these deviations from pure randomness. In a preferred embodiment, the symbolic transformation uses a length of the symbols k equal to 3 and a temporal separation ranging from 2 ms to 40 ms.

The weighted symbolic mutual information, representing the sharing of information across different brain areas, is calculated using said series of discrete symbols.

This information-theoretic metric presents three main advantages. First, weighted symbolic mutual information detects qualitative or “symbolic” patterns of increase or decrease in the signal, which allows a fast and robust estimation of the signals' entropies. Second, wSMI makes few hypotheses on the type of interactions and provides an efficient way to detect non-linear coupling. Third, the wSMI weights discard the spurious correlations between EEG signals arising from common sources and favor non-trivial pairs of symbols, as confirmed by simulations.

According to one embodiment, the wSMI is calculated in the theta frequency band (4-8 Hz) as the dominant resting state rhythms are typically observed at theta frequencies and this rhythm shows maximum changes in Alzheimer's disease patients.

According to one embodiment, the method comprises a further step consisting in the use of wSMI to estimate the functional connectivity (FC) between brain regions. Indeed, wSMI has proved to be effective in assessing FC because, unlike several traditional synchrony measures, it minimizes common-source artefacts and provides an efficient way to detect non-linear coupling. For wSMI, connectivity measures may be summarized by calculating the median value from each electrode to all the other electrodes.

The method may comprise a further step configured to compute functional connectivity matrices by calculating the mean of the wSMI values between electrodes belonging to different predefined clusters. Said predefined clusters of electrodes broadly define cortical regions: frontal right (FR) and left (FL), central right (CR) and left (CL), temporal right (TR) and left (TL), parietal right (PR) and left (PL) and occipital right (OR) and left (OL).

According to one embodiment, the method comprises a further step of computing intra and inter-hemispheric functional connectivity between parietal, temporal and occipital brain regions. It was found by the inventors that the inter-cluster functional connectivity between the clusters of electrodes, associated to the parietal, temporal and occipital brain regions, is significantly higher in preclinical Alzheimer's disease subjects compared to non-preclinical Alzheimer's disease subjects.

According to one embodiment, the power spectral density is extracted in the delta frequency band (1-4 Hz), theta frequency band (4-8 Hz), alpha frequency band (8-12 Hz), beta frequency band (12-30 Hz) and/or in the gamma frequency band (30-45 Hz). The power spectral density may be normalized.

The median spectral frequency may be further extracted as EEG metrics. The median spectral frequency (MSF) advantageously summarizes the relative distribution of power in the frequency spectrum and is therefore particularly efficient in the present case of preclinical Alzheimer's disease subjects which present opposing variations of low (delta) and higher (beta and gamma) frequencies.

According to one embodiment, the method further comprises a step configured to extract the spectral entropy (SE). The entropy of a time series is a measure of signal predictability and is thus a direct estimation of the information it contains. Spectral entropy basically quantifies the amount of organization of the spectral distribution. The spectral entropy may be calculated using the Shannon Entropy.

The method may further comprise a step configured to extract the algorithmic complexity, which estimates the complexity of an EEG signal based on its compressibility. The quantification of the complexity of EEG signals may be based on the application of the Kolmogorov-Chaitin complexity. This measure quantifies the algorithmic complexity of the signal acquired by a single EEG electrode by measuring his degree of redundancy.

An average across all epochs for each of the EEG metrics extracted across all electrodes may be computed.

Those EEG metrics advantageously allow to discriminate non-preclinical Alzheimer's disease subjects from preclinical Alzheimer's disease subjects, indeed the inventors found that neurodegeneration is associated to a significant widespread decrease of the power spectral density in the delta frequency band, a significantly higher fronto-central power spectral density in the beta and gamma frequency band, MSF, spectral entropy and algorithmic complexity.

According to one embodiment, the method comprises an evaluation step 103 consisting in the evaluation of the EEG metrics extracted and calculation of a neurodegeneration index.

According to one embodiment, the neurodegeneration index is calculated by comparison of at least one EEG metrics with at least one predefined threshold.

Each EEG metrics may be compared to a specific predefined threshold. Said predefined threshold may be defined in agreement with the trends observed by the inventors in the variation of the EEG metrics values between non-preclinical Alzheimer's disease subjects and preclinical Alzheimer's disease subjects.

The neurodegeneration index may be simply the deviation value between the EEG metrics and its predefined threshold or it may represent the probability that the subject has preclinical AD.

In one example, the functional connectivity in the theta band is compared with its predefined threshold for the differ brain region. Said comparison may be simply done by calculation of the difference between the functional connectivity value in the different brain regions and the predefined threshold, and averaging of these differences. In this example a positive neurodegeneration index will be obtained for preclinical Alzheimer's disease subjects, since the inventor have observed a widespread increase in functional connectivity in theta frequency band in preclinical Alzheimer's disease subjects.

The EEG metrics values may be combined in a mathematical function (e.g. a weighted function) in order to obtain a unique neurodegeneration index when multiple EEG matrices have been extracted.

The strength of the present invention is that the EEG metrics proposed are so adapted to represent the neurodegeneration cause by AD, even in its preclinical stage, that no complex and time consuming analysis process, requiring a comparison with large data base of clinical cases, is necessary to obtain a neurodegeneration index aiding the physician in making a reliable and early diagnosis of preclinical Alzheimer's disease only on the base of easily available EEG signals.

According to one embodiment, the neurodegeneration index is representative of the stage of preclinical Alzheimer's disease affecting the subject. Indeed, the inventors has advantageously observed that the early preclinical stage is characterized by an increase in brain oscillations and functional connectivity while the later preclinical stage is characterized by a slowing of brain oscillations and reduced functional connectivity with an EEG pattern getting close to the one observed in MCI and AD. Therefore, according to the range of values in which are comprised functional connectivity and the other EEG metrics, it is possible to provide a neurodegeneration index that guides the physician in the discrimination between early and late preclinical Alzheimer's disease stage.

According to one embodiment, the method 100 further comprises a step 104 of outputting the neurodegeneration index.

The present invention further relates to a computer program product for measuring and monitoring neurodegeneration in a subject, the computer program product comprising instructions which, when the program is executed by a computer, cause the computer to carry out the steps of the computer-implemented method for measuring and monitoring neurodegeneration of a subject according to any one of the embodiments described hereabove.

The present invention further relates to a computer-readable storage medium comprising instructions which, when the program is executed by a computer, cause the computer to carry out the steps of the computer-implemented method for measuring and monitoring neurodegeneration of a subject according to any one of the embodiments described hereabove.

Computer programs implementing the method of the present embodiments can commonly be distributed to users on a distribution computer-readable storage medium such as, but not limited to, an SD card, an external storage device, a microchip, a flash memory device and a portable hard drive. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

Another aspect of the present invention concerns a system comprising multiple modules configured to measure and monitor neurodegeneration of a subject.

According to one embodiment, the system and their modules comprises dedicated circuitry or a general purpose computer, configured for receiving the data and executing the steps of the method for measuring and monitoring neurodegeneration described in the embodiments here above. According to one embodiment, the system comprises a processor and the computer program of the present invention.

According to one embodiment, the system comprises an acquisition module configured to control the acquisition of subject electroencephalographic signals using an electroencephalography system comprising at least two electrodes (i.e. acquisition channels). The transmission of commands for the acquisition to the electroencephalogram and the reception of the recorded electroencephalographic signals may be done by wire or wireless. The system may comprise the electroencephalography system.

As a variant, the acquisition module may be exclusively configured to receive electroencephalographic signals. Said electroencephalographic signals may be received by the system in real time during the acquisition or acquired and stored in a medical database and transmitted to the system in a second time.

According to one embodiment, the electroencephalographic signals are acquired using electroencephalogram from at least two electrodes, positioned onto predetermined areas of the scalp of the subject in order to obtain a multi-channel electroencephalographic signal. According to one embodiment, the electroencephalographic signals are acquired by at least 2, 4, 8, 10, 15, 16, 17, 18, 19, 20, 21, 32, 64, 128 or 256 electrodes. According to one embodiment, the electrodes are placed on the scalp according to the 10-10 or 10-20 system, dense-array positioning or any other electrodes positioning known by the man skilled in the art. The electrodes montage may be unipolar or bipolar. In one example, the electrodes may be placed accordingly to the 10-20 system with locations Fp1, Fp2, F7, F3, Fz, F4, F8, T3, C3, Cz, C4, T4, T5, P3, Pz, P4, T6, O1, O2, A1 and A2. In said embodiment, various types of suitable headsets or electrode systems are available for acquiring such neural signals. Examples includes, but are not limited to: Epoc headset commercially available from Emotiv, Waveguard headset commercially available from ANT Neuro, Versus headset commercially available from SenseLabs, DSI 6 headset commercially available from Wearable sensing, Xpress system commercially available from BrainProducts, Mobita system commercially available from TMSi, Porti32 system commercially available from TMSi, ActiChamp system commercially available from BrainProducts and Geodesic system commercially available from EGI.

The electroencephalographic signals received may be obtained with a standard recording module with sampling frequency of at least 24 Hz, preferably 32 Hz, 64 Hz, 128 Hz, 250 Hz or any other sampling frequency known by the man skilled in the art.

According to one embodiment, the acquisition set-up comprises an amplifier unit for magnifying and/or converting the electroencephalographic signals from analog to digital format.

According to one embodiment, the system comprises a pre-processing module for pre-processing of the electroencephalographic signals in order to remove or reject noise according to the embodiments described above. According to one embodiment, the electroencephalographic signals are further pre-processed in order to remove or reject artefact.

According to one embodiment, the system of the present invention comprises a calculation module configured to extract at least one EEG metric representative of neurodegeneration according to the embodiment described above.

According to one embodiment, the system further comprises an evaluation module configured to evaluate the at least one EEG metric and extract a neurodegeneration index according to the embodiment described above.

According to one embodiment, the system further comprises a user interface module providing the neurodegeneration index as output.

The system and method of the present invention which uses EEG, a non-invasive, cheap and widely-available technique, could be used as a screening tool for identifying individuals at high risk of neurodegeneration and future cognitive decline. EEG could also help to specify if individuals are at an early preclinical Alzheimer's disease stage (with intermediate amyloid burden) or at a late preclinical Alzheimer's disease stage (with very high amyloid burden).

While various embodiments have been described and illustrated, the detailed description is not to be construed as being limited hereto. Various modifications can be made to the embodiments by those skilled in the art without departing from the true spirit and scope of the disclosure as defined by the claims.

### EXAMPLES

The present invention is further illustrated by the following examples.

### Example 1

Materials and Methods

Observational Study Design and Participants

Twenty individuals having severe neurodegeneration were selected based on low ¹⁸F-FDG PET metabolism in AD-signature regions, combined with subthreshold to very high amyloid burden measured by ¹⁸F-florbetapir PET, to target subjects at highest risk of future cognitive decline. A control group of 20 neurodegeneration negative subjects was selected based on high ¹⁸F-FDG PET metabolism in the cohort, combined with low amyloid standardized uptake value ratio (SUVR), to target subjects at very low risk of future conversion to Alzheimer's disease and of cognitive decline, despite their subjective memory complaint. The beta-amyloid load was evaluated using ¹⁸F-florbetapir PET SUVR as a continuous variable, as a potential continuous non-linear relationship between amyloid burden and EEG measures may exist. It was hypothesized that preclinical Alzheimer's disease subjects would present specific EEG patterns and functional connectivity differences compared to controls. Moreover, it was hypothesized that these EEG patterns would be modulated differently depending on the degree of severity of amyloid burden.

PET Acquisition and Processing

PET scans were acquired 50 min after injection of 370 MBq (10 mCi) ¹⁸F-florbetapir or 30 min after injection of 2 MBq/kg ¹⁸F-FDG. Reconstructed images were analysed with a predefined pipeline. An ¹⁸F-florbetapir-PET SUVR threshold was set at 0.7918 to dichotomize subjects into amyloid positive and negative groups. In the present study it was decided to evaluate amyloid burden as a continuous measure, rather than using a categorical approach, in order to assess the impact of various degrees of severity of amyloid burden on EEG metrics.

The same image-assessment pipeline was applied to measure brain glucose metabolism on ¹⁸F-FDG PET scans. Cortical metabolic indices were calculated in four bilateral regions of interest that are specifically affected by AD: posterior cingulate cortex, inferior parietal lobule, precuneus, and inferior temporal gyrus, and the pons was used as the reference region. Subjects were considered neurodegeneration positive if the mean ¹⁸F-FDG PET SUVR of the 4 AD-signature regions was below 2.27.

EEG Acquisition and Processing

EEG data were acquired with a high-density 256-channel EGI system (Electrical Geodesics Inc., USA) with a sampling rate of 250 Hz and a vertex reference. During the recording, patients were instructed to keep awake and relaxed, with their eyes closed in a quiet room. 60 seconds of eyes-closed resting-state recording were selected for the analysis. For EEG data processing was used the pipeline that automates processing of EEG recordings with automated artefact removal and extraction of EEG measures.

The automated EEG data processing workflow was the following: EEG recordings were band-pass filtered (using a Butterworth 6th order high pass filter at 0.5 Hz and a Butterworth 8th order low pass filter at 45 Hz). A notch filter was applied at 50 Hz and 100 Hz. Data were cut into 1 second epochs with random separations between 10 and 100 milliseconds between them Channels that exceeded a 100 μv peak-to-peak amplitude in more than 50% of the epochs were rejected. Channels that exceeded a z-score of four across all the channels mean variance were rejected. This step was repeated two times. Epochs that exceeded a 100 μv peak-to-peak amplitude in more than 10% of the channels were rejected. Channels that exceeded a z-score of four across all the channels mean variance (filtered with a high pass of 25 Hz) were rejected. This step was repeated two times. The remaining epochs were digitally transformed to an average reference. Rejected channels were interpolated.

Calculation and Analysis of the EEG Metrics

The set of 40 high-density 256-channel EEG recordings were analyzed. For each recording, we extracted a set of measures organized according to a theory-driven taxonomy, as described by (Sitt et al., 2014). In total, 10 EEG metrics were calculated: power spectral density in delta (1-4 Hz), theta (4-8 Hz), alpha (8-12 Hz), beta (12-30 Hz), gamma (30-45 Hz), median spectral frequency, spectral entropy, algorithmic complexity and wSMI in theta and alpha band. The 10 EEG metrics were averaged across all epochs (60 seconds recording) and power spectral density was normalized.

EEG Metrics Analysis

To study the impact of group, age, gender, educational level, apolipoprotein E4 (ApoE4) status and ¹⁸F-florbetapir SUVR on EEG metrics, two types of analyses were performed. The first one concerned calculation of the value of each metric for each electrode so that each participant was associated to 256 values for each metric. For wSMI, connectivity measures were summarized by calculating the median value from each electrode to all the other electrodes. The second analysis was on the mean value of each metric across all electrodes.

First for each analysis simple models were performed to test main effects one by one. If the effect was significant at level 0.10 for at least one EEG metric, it was included in multiple models. Then multiple models were performed to evaluate main effects together. P-values were corrected for multiple testing on 10 measures with the Benjamini-Hochberg False discovery rate (BH-FDR) procedure. Models were validated checking normal distribution of residuals, Cook's distance and absence of heteroskedasticity. For the analysis of the mean value of each metric across all electrodes, linear regression was performed. For the analysis of the value of each metric at each electrode, linear mixed models were performed with the effect of interest as fixed effect as well as the electrode number and the subject as random effect. Interactions between electrode number and main effects were tested one by one. Type II tests were performed. When an interaction was significant, post hoc tests were performed at electrode level, to identify the most relevant electrodes for discriminating between groups for a given EEG metric. Because of the small sample size and exploratory nature of this study, we did not correct post hoc tests for multiplicity on 256 electrodes. We generated scalp topographical maps using FieldTrip MATLAB software toolbox.

Comparison of FC Matrices Between Groups

To ease interpretation of the large number of channels, were used 10 clusters of electrodes used, which broadly define cortical regions. It was computed the average wSMI between each region by calculating the mean of all the wSMI that the electrodes of one region shared with all the electrodes of another region and produced functional connectivity matrices. We used a linear mixed model to compare the inter-cluster wSMI average values between the two groups. Interaction between group and inter-cluster mean wSMI was tested. When an interaction was significant post-hoc tests were performed to identify the most relevant inter-cluster connections that significantly differed in weights between groups.

All p-values are adjusted on age, educational level, gender, ApoE4 status and ¹⁸F-florbetapir SUVR. P-values were reported as significant if less than 0.05.

Results

Population Baseline Characteristics Analysis

The mean age of all participants was 76.6 years (SD 4.3) and the educational level was high as show in Table 1. No significant differences were present in age and educational level between the two groups. There were significantly more women in the control group and more men in the preclinical Alzheimer's disease group. The proportion of ApoE4 carriers was higher in the preclinical Alzheimer's disease group than in the control group (35% versus 5% respectively). The two groups did not differ for cognitive scores except for the “Free and clued selective reminding test” delayed free recall where the preclinical Alzheimer's disease group had significantly lower scores (P=0.001).

TABLE 1

| | All participants | Control group | Preclinical AD group | |
|---|---|---|---|---|
| | (n = 40) | (n = 20) | (n = 20) | p-value* |
| Demographics | | | | |
| Age (years) | 76.6 ± 4.3 | 76.1 ± 4.1 | 77.2 ± 4.5 | 0.407 |
| Men | 19 (47.50%) | 4 (20.00%) | 15 (75.00%) | <0.001* |
| Women | 21 (52.50%) | 16 (80.00%) | 5 (25.00%) | — |
| High educational level§ | 26 (65.00%) | 12 (60.00%) | 14 (70.00%) | 0.507 |
| APOE ε4 allele | 8 (20.00%) | 1 (5.00%) | 7 (35.00%) | 0.018* |
| Cognitive tests | | | | |
| Mini-Mental State Examination | 28.650 ± 0.949 | 28.750 ± 1.070 | 28.550 ± 0.826 | 0.512 |
| Free and Cued Selective Reminding Test | | | | |
| Immediate Free Recall | 28.475 ± 5.657 | 29.450 ± 6.236 | 27.500 ± 4.979 | 0.281 |
| Immediate Total Recall | 45.825 ± 2.011 | 46.000 ± 2.152 | 45.650 ± 1.899 | 0.589 |
| Delayed Free Recall | 10.800 ± 2.441 | 12.000 ± 2.224 | 9.600 ± 2.062 | 0.001* |
| Delayed Total Recall | 15.425 ± 0.874 | 15.550 ± 0.686 | 15.300 ± 1.031 | 0.372 |
| Frontal Assessment Battery | 16.359 ± 1.724 | 16.650 ± 1.663 | 16.053 ± 1.779 | 0.285 |
| ¹⁸F-fluorodeoxyglucose PET imaging | | | | |
| Mean FDG Standardized uptake value ratios† | 2.496 ± 0.451 | 2.924 ± 0.136 | 2.068 ± 0.121 | <0.001* |
| ¹⁸F-florbetapir PET imaging | | | | |
| Standardized uptake value ratios | 0.841 ± 0.242 | 0.682 ± 0.053 | 1.000 ± 0.254 | <0.001* |
| Volumetric MRI (cm³) | | | | |
| Total hippocampal volume¶ | 2.687 ± 0.228 | 2.826 ± 0.177 | 2.549 ± 0.188 | <0.001* |

The mean ¹⁸F-FDG PET SUVR was 2.068 (SD 0.121) in the preclinical Alzheimer's disease group and 2.924 (SD 0.136) in the control group. The mean cortical SUVR for ¹⁸F-florbetapir PET was significantly higher in the preclinical Alzheimer's disease group than in the control group, with values of 1.000 (SD 0.254) and 0.682 (SD 0.053) respectively. The total hippocampal volume measured on structural MRI was significantly lower in preclinical Alzheimer's disease subjects compared to controls (P<0.001).

256 Electrodes Analysis: Topographical Differences Across EEG Measures and Groups

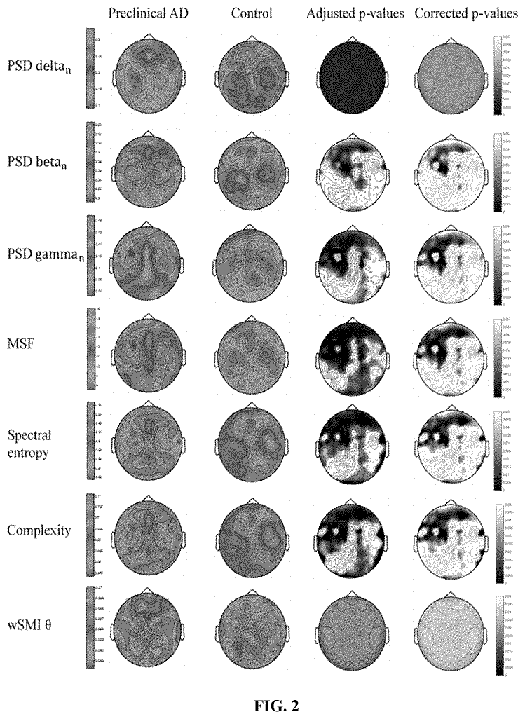

Several power spectrum measures were efficient indices in discriminating preclinical Alzheimer's disease subjects from controls (FIG. 2 and Table 2). As age and level of education had no significant impact on EEG metrics in a simple model, p-values were adjusted on ApoE4 status, gender and amyloid SUVR. Preclinical Alzheimer's disease subjects presented a significant widespread delta power decrease compared to controls (P=0.008, FDR-corrected P=0.030). Beta and gamma power were significantly higher in fronto-central regions in the preclinical Alzheimer's disease group compared to controls (P=0.028, FDR-corrected P=0.040 and P=0.016, FDR-corrected P=0.032, respectively). Theta and alpha power failed to discriminate between groups.

Because of these opposing variations of low (delta) and higher (beta and gamma) frequencies, the median spectral frequency (MSF), which summarizes the relative distribution of power in the frequency spectrum, was particularly efficient. MSF was significantly higher in fronto-central regions in preclinical Alzheimer's disease subjects compared to controls (P=0.003, FDR-corrected P=0.03). Preclinical Alzheimer's disease subjects presented a higher spectral entropy in fronto-central regions, meaning a less predictable spectral structure, than the controls (P=0.014, FDR-corrected P=0.032). Algorithmic complexity was significantly higher in fronto-central regions in the preclinical Alzheimer's disease group compared to controls (P=0.009, FDR-corrected P=0.03).

Measures of functional connectivity based on information theory were particularly efficient for discriminating between the two groups. In preclinical Alzheimer's disease and control subjects, topographical analysis showed that mesio-parietal areas were the maximally connected regions to the rest of the brain. Preclinical Alzheimer's disease subjects presented a significant widespread increase of wSMI in theta band compared to controls (P=0.028, FDR-corrected P=0.040). There was no significant difference for wSMI in alpha band between the two groups.

Mean Value of Each EEG Metric Across All Electrodes

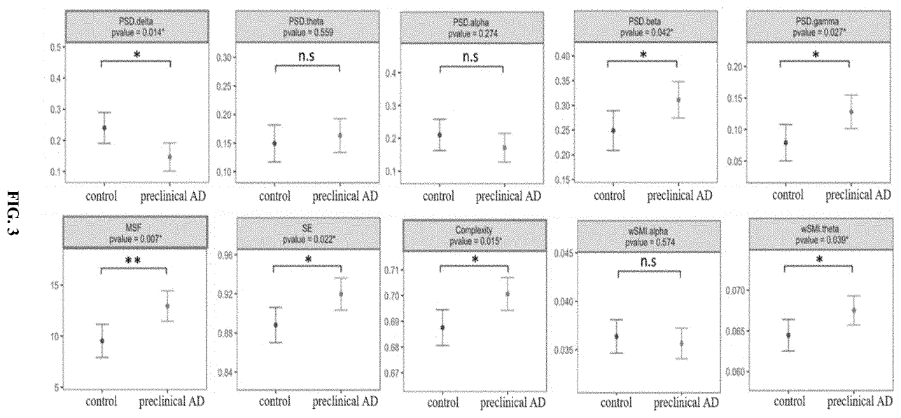

To reduce dimensionality, we summarized spatial information by considering the average of each EEG metric over all scalp electrodes (FIG. 3 and Table 3). The aim was to assess the discrimination capacity of the mean value of each EEG metric between controls and preclinical Alzheimer's disease subjects. In case of good discriminative power, it would mean that only the average value of EEG metrics across all electrodes would need to be used to further classify subjects in the preclinical Alzheimer's disease or the control group, without needing to analyze 256 values for each metric which would avoid the problem of multiple comparisons on many electrodes. This could be particularly important for implementing this marker in clinical practice. We report Cohen's f2 values to indicate effect size for each metric (Cohen J. Statistical Power Analysis for the Behavioral Sciences. Elsevier; 1988.). P-values were adjusted on ApoE4 status, gender and amyloid SUVR.

TABLE 2

| | Group | | | Interaction Electrode.Group | | |
|---|---|---|---|---|---|---|
| | | Adjusted | Corrected | | Adjusted | Corrected |
| EEG metrics | Chisq | p-value | p-value | Chisq | p-value | p-value |
| PSD deltaₙ | 7.02 | 0.008** | 0.030* | 0.74 | 0.999 | 1.000 |
| PSD thetaₙ | 0.3 | 0.587 | 0.587 | 2.68 | <0.001*** | <0.001*** |
| PSD alphaₙ | 1.2 | 0.274 | 0.343 | 1.45 | <0.001*** | <0.001*** |
| PSD betaₙ | 4.83 | 0.028* | 0.040* | 1.36 | <0.001*** | <0.001*** |
| PSD gammaₙ | 5.82 | 0.016* | 0.032* | 1.84 | <0.001*** | <0.001*** |
| MSF | 8.64 | 0.003** | 0.030* | 1.34 | <0.001*** | <0.001*** |
| Spectral entropy | 6.08 | 0.014* | 0.032* | 1.69 | <0.001*** | <0.001*** |
| Complexity | 6.76 | 0.009** | 0.030* | 1.33 | <0.001*** | <0.001*** |
| wSMI theta | 4.81 | 0.028* | 0.040* | 0.97 | 0.632 | 0.790 |
| wSMI alpha | 0.3 | 0.583 | 0.587 | 0.66 | 1.000 | 1.000 |

Participants from the preclinical Alzheimer's disease group had significantly lower delta power (P=0.014) and higher beta and gamma power (P=0.042 and P=0.027, respectively). MSF, spectral entropy, complexity and wSMI in theta band were significantly higher in the preclinical Alzheimer's disease group compared to controls (P=0.007, P=0.022, P=0.015 and P=0.039, respectively). In our study the average EEG metrics with the higher effect size were MSF (f2=0.235), delta power (f2=0.189), complexity (f2=0.188), spectral entropy (f2=0.165) and gamma power (f2=0.152), which corresponds to a medium effect size according to Cohen's guidelines. wSMI in theta band and beta power had a small effect size according to Cohen's guidelines (f2=0.131 and f2=0.127, respectively).

After correcting for multiple comparisons, delta power remained significantly lower in the preclinical Alzheimer's disease group (FDR-corrected P=0.049) and MSF and complexity remained significantly higher in the preclinical Alzheimer's disease group (FDR-corrected P=0.049 and FDR-corrected P=0.049, respectively) compared to controls. The other EEG metrics did not remain significant after multiple comparison correction.

Relationship Between Average EEG Metrics and Amyloid SUVR, ApoE4 Status and Gender

Multiple linear regression was used to study the relationship between the average measures of EEG metrics across all electrodes and several predictor variables. Predictor variables included in the multiple model were the following: group (as described previously), ApoE4 status, gender and ¹⁸F-florbetapir SUVR Table 3. Table 3 shows the results of multiple linear regression analysis for all explanatory variables for average EEG measures across all electrodes. R-squared values, Cohen's effect size f2, beta coefficient estimate±standard error, t-values, p-values and Benjamini-Hochberg corrected p values are shown. *P<0.05, **P<0.01, ***P<0.001. AD=Alzheimer's disease; ApoE=Apolipoprotein E; MSF=median spectral frequency; SUVR=standardized uptake value ratio; wSMI=weighted symbolic mutual information.

TABLE 3

| | | | | Beta estimate ± | | Adjusted | Corrected |
|---|---|---|---|---|---|---|---|
| EEG metrics | | R² | f2 | Standard Error | t value | p-value | p-value |
| wSMI theta | (Intercept) | 0.466 | . . . | 0.0646 ± 0.0020 | 33.087 | <0.001 | . . . |
| | Amyloid SUVR | | 0.027 | −0.0026 ± 0.0027 | −0.978 | 0.335 | 0.419 |
| | ApoE4+ | | 0.038 | 0.0014 ± 0.0013 | 1.148 | 0.259 | 0.647 |
| | Preclinical AD | | 0.131 | 0.0031 ± 0.0014 | 2.143 | 0.039* | 0.060 |
| | group | | | | | | |
| | Gender (male) | | 0.168 | 0.0027 ± 0.0011 | 2.427 | 0.021* | 0.205 |
| wSMI alpha | (Intercept) | 0.170 | . . . | 0.0339 ± 0.0017 | 19.614 | <0.001 | . . . |
| | Amyloid SUVR | | 0.006 | 0.0011 ± 0.0023 | 0.452 | 0.654 | 0.654 |
| | ApoE4+ | | 0.060 | 0.0016 ± 0.0011 | 1.452 | 0.574 | 0.518 |
| | Preclinical AD | | 0.009 | −0.0008 ± 0.0013 | −0.568 | 0.574 | 0.574 |
| | group | | | | | | |
| | Gender (male) | | 0.089 | 0.0017 ± 0.0010 | 1.762 | 0.087 | 0.434 |
| PSD deltaₙ | (Intercept) | 0.345 | . . . | 0.1479 ± 0.0497 | 2.978 | 0.005 | . . . |
| | Amyloid SUVR | | 0.153 | 0.1559 ± 0.0673 | 2.317 | 0.027* | 0.044* |
| | ApoE4+ | | 0.104 | −0.0603 ± 0.0317 | −1.904 | 0.065 | 0.326 |
| | Preclinical AD | | 0.189 | −0.0934 ± 0.0363 | −2.573 | 0.014* | 0.049* |
| | group | | | | | | |
| | Gender (male) | | 0.012 | −0.0177 ± 0.0277 | −0.639 | 0.527 | 0.937 |
| PSD alphaₙ | (Intercept) | 0.168 | . . . | 0.1134 ± 0.0482 | 2.354 | 0.024 | . . . |
| | Amyloid SUVR | | 0.040 | 0.0771 ± 0.0652 | 1.181 | 0.246 | 0.351 |
| | ApoE4+ | | 0.326 | 0.0593 ± 0.0307 | 1.932 | 0.062 | 0.107 |
| | Preclinical AD | | 0.035 | −0.0392 ± 0.0352 | −1.112 | 0.274 | 0.342 |
| | group | | | | | | |
| | Gender (male) | | 0.001 | 0.0052 ± 0.0269 | 0.193 | 0.848 | 0.937 |
| PSD betaₙ | (Intercept) | 0.230 | . . . | 0.3741 ± 0.0403 | 9.284 | <0.001 | . . . |
| | Amyloid SUVR | | 0.215 | −0.1497 ± 0.0546 | −2.742 | 0.010** | 0.024* |
| | ApoE4+ | | 0.001 | −0.0056 ± 0.0257 | −0.219 | 0.828 | 0.828 |
| | Preclinical AD | | 0.127 | 0.0621 ± 0.0295 | 2.108 | 0.042* | 0.060 |
| | group | | | | | | |
| | Gender (male) | | 0.003 | 0.0071 ± 0.0225 | 0.317 | 0.753 | 0.937 |
| PSD thetaₙ | (Intercept) | 0.147 | . . . | 0.1109 ± 0.0324 | 3.424 | 0.002 | . . . |
| | Amyloid SUVR | | 0.020 | 0.0370 ± 0.0439 | 0.844 | 0.405 | 0.450 |
| | ApoE4+ | | 0.017 | 0.0161 ± 0.0207 | 0.779 | 0.442 | 0.813 |
| | Preclinical AD | | 0.010 | 0.0140 ± 0.0237 | 0.590 | 0.559 | 0.574 |
| | group | | | | | | |
| | Gender (male) | | 0.000 | −0.0014 ± 0.0181 | −0.080 | 0.937 | 0.937 |
| PSD gammaₙ | (Intercept) | 0.233 | . . . | 0.1621 ± 0.0290 | 5.587 | <0.001 | . . . |
| | Amyloid SUVR | | 0.180 | −0.0988 ± 0.0393 | −2.513 | 0.017* | 0.033* |
| | ApoE4+ | | 0.003 | −0.0061 ± 0.0185 | −0.329 | 0.744 | 0.828 |
| | Preclinical AD | | 0.152 | 0.0490 ± 0.0212 | 2.309 | 0.027* | 0.054 |
| | group | | | | | | |
| | Gender (male) | | 0.004 | 0.0060 ± 0.0162 | 0.372 | 0.712 | 0.937 |
| Spectral | (Intercept) | 0.276 | | 0.9518 ± 0.0180 | 52.905 | <0.001 | |
| entropy | Amyloid SUVR | | 0.272 | −0.0753 ± 0.0244 | −3.087 | 0.004** | 0.013* |
| | ApoE4+ | | 0.003 | −0.0038 ± 0.0115 | −0.327 | 0.746 | 0.828 |
| | Preclinical AD | | 0.165 | 0.0316 ± 0.0132 | 2.404 | 0.022* | 0.054 |
| | group | | | | | | |
| | Gender (male) | | 0.003 | 0.0031 ± 0.0101 | 0.307 | 0.761 | 0.937 |
| MSF | (Intercept) | 0.317 | . . . | 14.6790 ± 1.6290 | 9.011 | <0.001 | . . . |
| | Amyloid SUVR | | 0.270 | −6.7914 ± 2.2073 | −3.077 | 0.004** | 0.013* |
| | ApoE4+ | | 0.014 | 0.7286 ± 1.0389 | 0.701 | 0.487 | 0.813 |
| | Preclinical AD | | 0.235 | 3.4179 ± 1.1908 | 2.870 | 0.007** | 0.049* |
| | group | | | | | | |
| | Gender (male) | | 0.006 | 0.4137 ± 0.9101 | 0.455 | 0.652 | 0.937 |
| Complexity | (Intercept) | 0.303 | . . . | 0.7103 ± 0.0070 | 101.901 | <0.001 | . . . |
| | Amyloid SUVR | | 0.275 | −0.0293 ± 0.0095 | −3.100 | 0.004** | 0.013* |
| | ApoE4+ | | 0.002 | 0.0011 ± 0.0044 | 0.239 | 0.812 | 0.828 |
| | Preclinical AD | | 0.188 | 0.0131 ± 0.0051 | 2.564 | 0.015* | 0.049* |
| | group | | | | | | |
| | Gender (male) | | 0.013 | 0.0026 ± 0.0039 | 0.678 | 0.502 | 0.937 |

No significant relationship was found between ApoE4 status and EEG metrics' average values. Concerning gender, average wSMI in theta band was significantly higher in men than in women (P=0.021), however this result did not remain significant after FDR correction.

No significant relationship was found between gender and the other EEG metrics. 256 electrodes topographical analysis of EEG metrics according to gender and ApoE4 showed similar results. There was a significant positive relationship between amyloid SUVR and delta power (P=0.026, FDR-corrected P=0.044), meaning that when amyloid SUVR values increased, delta power increased. There was a significant negative relationship between amyloid SUVR and beta power (P=0.010, FDR-corrected P=0.024), gamma power (P=0.017, FDR-corrected P=0.033), spectral entropy (P=0.004, FDR-corrected P=0.013), MSF (P=0.004, FDR-corrected P=0.013) and complexity (P=0.004, FDR-corrected P=0.013), meaning that when amyloid SUVR values increased the mean value of these EEG metrics decreased (Table 3).

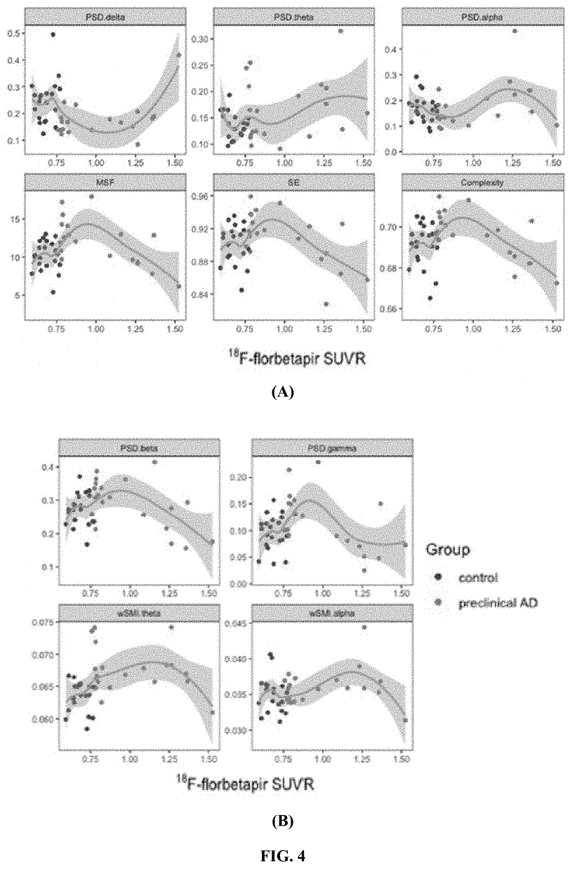

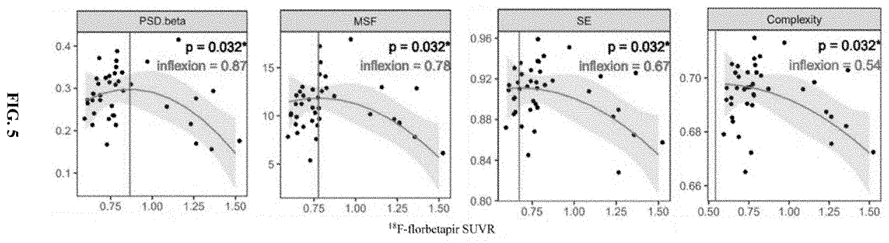

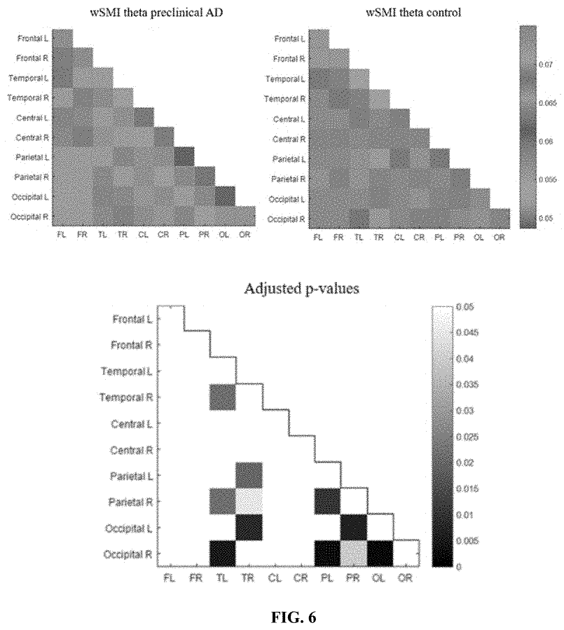

It was decided to complete this analysis using a local regression (LOESS) as the relation between amyloid SUVR and EEG metrics seemed complex and a non-linear model would probably better fit the data (FIGS. 4A and 4B). The relationship between amyloid SUVR and delta power followed a U-shape curve whereas the relationship between amyloid SUVR and beta and gamma power, MSF, spectral entropy, complexity and wSMI in theta band followed an inverted U-shape curve. It was used multiple regression with linear and quadratic effect of amyloid SUVR to determine its inflection points. They are displayed in FIG. 5, for the four EEG metrics that stayed statistically significant with this last regression model. Amyloid SUVR inflection value was 0.87 for beta power, 0.78 for MSF and 0.67 for spectral entropy. For complexity, the inflection point (0.54) was not interpretable as it was lower than the lowest amyloid SUVR value (0.594) among the 40 subjects.

Comparison of FC Matrices Between Groups

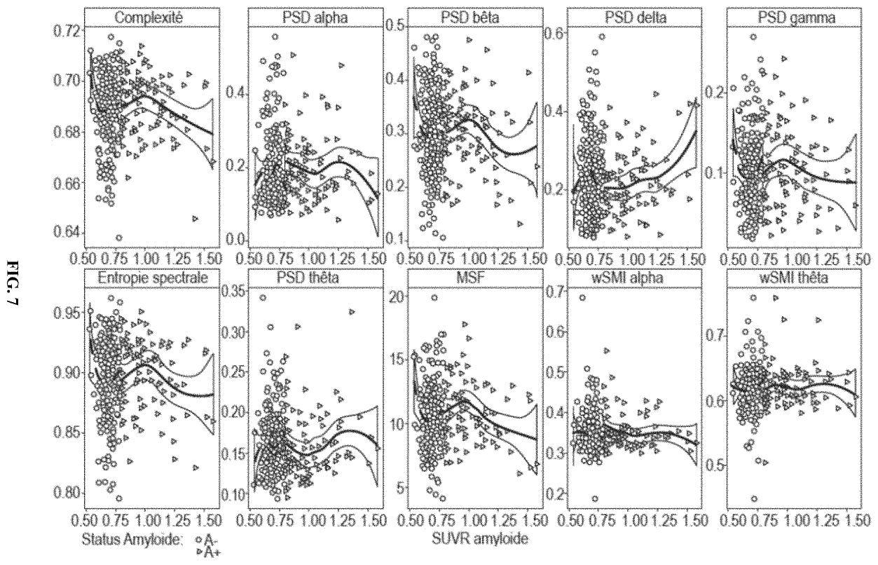

It was analyzed the inter-cluster functional connectivity between 10 clusters of electrodes, each cluster broadly defining a cortical region (FIG. 6): frontal right (FR) and left (FL), central right (CR) and left (CL), temporal right (TR) and left (TL), parietal right (PR) and left (PL) and occipital right (OR) and left (OL). P-values were adjusted on gender, ApoE4 status and amyloid SUVR. There was no main effect of group but there was a significant interaction between group and inter-cluster functional connectivity (P<0.001). Post-hoc analysis revealed that the following inter-cluster connections had significantly higher weights in preclinical Alzheimer's disease subjects compared to controls: OL-OR (P=0.002), PL-OR (P=0.003), PL-PR (P=0.011), PR-OL (P=0.007), TR-OL (P=0.008), TR-PR (P=0.045), TL-OR (P=0.005), TL-PR (P=0.022), TL-TR (P=0.022), TR-PL (P=0.02) and PR-OR (P=0.04). To sum up, intra and inter-hemispheric FC between parietal, temporal and occipital brain regions was significantly higher in preclinical Alzheimer's disease subjects compared to controls. However, none of these values remained significant after multiplicity correction on 55 inter-cluster connections.

Discussion

At the knowledge of the Applicant, this was the first study to demonstrate EEG changes in preclinical AD. In addition, it links these changes to compensatory mechanisms at this early stage of the disease. Moreover, it was explored the combined effect of neurodegeneration and amyloid-beta deposition on EEG metrics, treating amyloid burden as a continuous variable.

Neurodegeneration was associated to a significant widespread delta power decrease, a significantly higher fronto-central beta and gamma power, MSF, spectral entropy and algorithmic complexity. Another striking difference between groups was a widespread increase in FC in theta frequency band (wSMI theta) in preclinical Alzheimer's disease subjects compared to controls. Importantly, the vigilance level did not differ between groups, as confirmed by the absence of EEG sleep figures after blinded visual analysis of the 40 EEG recordings by two neurologists and a similar number of artefacts in the two groups.

A most interesting result is the evidence of a non-linear relationship between amyloid burden and EEG metrics, either following a U-shape curve for delta power or an inverted U-shape curve for the other metrics, meaning that EEG patterns are modulated differently depending on the degree of severity of amyloid burden. More precisely, we found that before preclinical Alzheimer's disease subjects exceed a certain amyloid load, the trend of their EEG metrics is similar to the one that is observed at the whole preclinical Alzheimer's disease group level analysis, as described previously, meaning lower delta power and higher beta and gamma power, MSF, spectral entropy, algorithmic complexity and wSMI in theta band. However, after preclinical Alzheimer's disease subjects exceed a certain threshold of amyloid load, the whole trend of EEG metrics reverses, meaning increased delta power and decreased beta and gamma power, MSF, spectral entropy, algorithmic complexity and wSMI in theta band. It is interesting to notice that the amyloid SUVR inflection point found in the present study for MSF is 0.78) is very close to the threshold of 0.79 set for positive versus negative Aβ deposition in observational study, as reported by (Dubois et al. Lancet Neurol 2018; 17: 335-346; Habert et al., Annals of Nuclear Medicine 2018; 32: 75-86) and that the inflection point for beta power (0.87) is very close to the more stringent threshold of 0.88 set to determine amyloid positivity also in observational study as reported by (Teipel et al., 2018, Neuroimage Clin 2018; 17: 435-443). Our results indicate that two different EEG stages can be differentiated in preclinical AD: an early and a late stage, depending on the severity of amyloid burden.

Focusing first on the results for the first phase of preclinical AD, before amyloid load exceeds a critical threshold. Increasing high frequency spectral power in fronto-central regions is in line with one recent study which showed a functional frontal upregulation revealed by an increased frontal alpha power in preclinical Alzheimer's disease subjects (Nakamura et al., Brain 2018; 141: 1470-1485). Compared to this previous study, we found a frontal upregulation in higher frequency bands which were beta (12-30 Hz) and gamma (30-45 Hz). Increased frontal functional upregulation has also been shown in other studies with an increased FC in frontal regions (Mormino et al., Cerebral Cortex 2011; 21: 2399-2407; Jones et al., Brain 2016; 139: 547-562). In an inverse way we found a widespread decrease in delta power in preclinical Alzheimer's disease subjects, before amyloid load goes beyond an excessive burden. To the Applicant knowledge, this is the first study to show a decrease in low-frequency oscillations in preclinical Alzheimer's disease subjects. The first hypothesis to explain an increase in frontal high-frequency oscillations concomitant with a decrease in low-frequency oscillations in the early phase of preclinical Alzheimer's disease is a compensatory mechanism, which was also proposed in previous studies (Mormino et al., Cerebral Cortex 2011; 21: 2399-2407; Lim et al., Brain 2014; 137: 3327-3338; Jones et al., Brain 2016; 139: 547-562). A sufficient level of compensation is needed to maintain normal cognitive function despite amyloid burden and hypometabolism in preclinical AD. Compensatory mechanisms would then fail once amyloid burden exceeds a certain level, explaining the reversal of the EEG metrics trend, with a slowing of brain oscillations revealed by increased delta power and decreased beta and gamma power, with a spectral pattern getting close to the one typically found in MCI and AD. Another explanation is that as participants in observational study are selected on normal cognition, subjects with neurodegeneration and high amyloid load may have a particularly high cognitive reserve, which is revealed by baseline higher spectral power in frontal regions, reduced low-frequency oscillations and higher FC (Cohen et al., Journal of Neuroscience 2009; 29: 14770-14778; Mormino et al., Cerebral Cortex 2011; 21: 2399-2407; Lim et al., Brain 2014; 137: 3327-3338); this cognitive reserve would be altered as amyloid load increases, which would explain why subjects with high neurodegeneration and very high amyloid load show slowing of brain oscillations and lower FC.

Another hypothesis is abnormal transient neuronal hyperexcitability related to Aβ deposition with a relative decrease in synaptic inhibition (Busche et al., Science 2008; 321: 1686-1689; Palop and Mucke, Nature Neuroscience 2010; 13: 812-818; Nakamura et al., Brain 2018; 141: 1470-1485). A histological study by (Garcia-Marin, Front Neuroanat 2009; 3: 28) showed diminished GABAergic terminals near amyloid plaques. It could explain the increase in high-frequency oscillations and enhanced FC in temporo-parieto-occipital regions which are areas with high amyloid burden.

The ‘acceleration’ hypothesis suggests that once Aβ deposition is initiated by independent events, a milieu of higher FC hastens this deposition, which eventually leads to the functional disconnection or metabolic deterioration in the subjects with amyloid burden (Cohen et al., Journal of Neuroscience 2009; 29: 14770-14778; de Haan et al., PLoS Comput Biol 2012; 8: e1002582; Johnson et al., Neurobiology of Aging 2014; 35: 576-584; Lim et al., Brain 2014; 137: 3327-3338). During this period, there might be possibilities of the toxic excitation of affected neurons and compensatory higher FC induced by the amyloid retention (Mormino et al., Cerebral Cortex 2011; 21: 2399-2407). The metabolic demands associated with high connectivity may be the detrimental phenomenon that triggers downstream cellular and molecular events associated with Alzheimer's disease (Jones et al., Brain 2016; 139: 547-562). Previous work in animal models has shown that intermediate levels of Aβ enhance synaptic activity presynaptically (Abramov et al., Nature Neuroscience 2009; 12: 1567), whereas abnormally high levels of Aβ impair synaptic activity by inducing post-synaptic depression (Palop and Mucke, Nature Neuroscience 2010; 13: 812-818). This is consistent with our results showing basically two different EEG phases in preclinical AD. In the early preclinical stage that is characterized by neurodegeneration combined with intermediate levels of Aβ, there is an increase in brain oscillations and FC due to compensation and/or Aβ related excitotoxicity. Then, FC increase would hasten Aβ deposition. In a later preclinical stage characterized by neurodegeneration combined with very high levels of Aβ, there is a slowing of brain oscillations and reduced FC due to compensatory mechanisms failure and/or post-synaptic depression, with an EEG pattern getting close to the one observed in MCI and AD.

Inter-region connectivity analysis showed that FC was specifically increased between parietal, temporal and occipital regions in the preclinical Alzheimer's disease group. These regions partially overlap with some key regions of the DMN, as posterior cingulate cortex and inferior parietal cortex have been described as important hubs in the DMN (Miao et al., PLoS ONE 2011; 6: e25546). Similar results were found in some recent preclinical Alzheimer's disease studies, with increased FC in the DMN (Lim et al., Brain 2014; 137: 3327-3338) and increased FC between the precuneus and the bilateral parietal lobules in cognitively normal amyloid positive subjects while a local decrease in FC has been found within the precuneus (Nakamura et al., Scientific Reports 2017; 7: 6517). This raised the hypothesis of locally disrupted FC by Aβ deposition, compensated by higher connectivity in medium to long range networks. A cascading network failure has been proposed by (Jones et al., Brain 2016; 139: 547-562), with a failure beginning in the posterior DMN which then shifts processing burden to other systems containing prominent connectivity hubs. This posterior DMN decline is accompanied by transient increased connectivity between the posterior DMN and other brain systems and is quantified in the recently developed neuromarkers termed the Network Failure Quotient (Wiepert et al., Alzheimer's & Dementia: Diagnosis, Assessment & Disease Monitoring 2017; 6: 152-161). The break-down of the initial functional compensation would facilitate accelerated tau-related neurodegenerative processes (Jones et al., Cortex 2017; 97: 143-159).

To the applicant knowledge this example is the first to study complexity and spectral entropy in preclinical Alzheimer's disease subjects, coupled with metabolic evidence of neurodegeneration and Aβ biomarker information. The increased complexity and spectral entropy observed in early preclinical Alzheimer's disease in frontal areas could also be explained by compensatory mechanisms. Compensation would then fail in a later stage of preclinical AD, with an EEG pattern becoming less complex and more regular, getting close to the one observed in MCI and Alzheimer's disease (Hornero et al., Philosophical Transactions of the Royal Society A: Mathematical, Physical and Engineering Sciences 2009; 367: 317-336; Staudinger and Polikar, IEEE; 2011. p. 2033-2036; Al-Nuaimi et al., Complexity 2018; 2018: 1-12).

Another novelty of our example is the selection of our study population on a neurodegeneration criterion in contrast to the more commonly used selection of individuals at risk for Alzheimer's disease based on amyloid biomarker alone with a dichotomous classification of subjects as amyloid-negative or positive. First, amyloid deposition alone does not necessarily represent progression to Alzheimer's disease as both neuropathological and PET data show evidence of extensive amyloid-β pathology in cognitively normal older people (Bennett et al., Neurology 2006; 66: 1837-1844; Morris et al., Annals of Neurology 2010; 67: 122-131; Jagust, Brain 2016; 139: 23-30).

Second, dichotomous treatment of a continuous variable, such as Aβ, potentially obscures the true relationship of amyloid burden with EEG metrics. Third, it has been shown that neurodegeneration, particularly synapse loss, is the aspect of Alzheimer's disease neuropathologic change that correlates most closely with symptom onset and cognitive decline (Soldan et al., JAMA Neurology 2016; 73: 698; Jack et al., Alzheimer's & Dementia 2018; 14: 535-562) and several studies using FDG-PET showed that cerebral metabolic rate of glucose reduction predicted cognitive decline from normal elderly cognition to MCI/AD with a high accuracy, decliners showing greater reduction of PET-FDG SUVR values (de Leon et al., Proceedings of the National Academy of Sciences 2001; 98: 10966-10971; Jagust et al., Annals of Neurology 2006; 59: 673-681; Mosconi et al., European Journal of Nuclear Medicine and Molecular Imaging 2009; 36: 811-822, Mosconi et al., Journal of Alzheimer's Disease 2010; 20: 843-854). Thus, our selection procedure maximized our chances of identifying subjects at a preclinical Alzheimer's disease stage, with a high risk of cognitive decline.

ApoE4 status did not have any significant impact on EEG metrics. This is consistent with some previous EEG studies on cognitively normal subjects which did not find any differences according to ApoE genotype neither for spectral patterns (Ponomareva et al., Neurobiology of Aging 2008; 29: 819-827; Jiang et al., Neuroscience Letters 2011; 505: 160-164) nor for FC (Bassett et al., Brain 2006; 129: 1229-1239; Nakamura et al., Scientific Reports 2017; 7: 6517), whereas some studies found higher alpha synchronization likelihood (Kramer et al., Clinical Neurophysiology 2008; 119: 2727-2732) or reduced brain activity in ApoE4 carriers (Lind et al., Brain 2006; 129: 1240-1248). We found that men had higher posterior FC; however, this result should be interpreted with caution as there was some gender imbalance between groups. Some studies have found higher FC in men (Allen et al., Frontiers in Systems Neuroscience 2011; 5:2; Filippi et al., Human Brain Mapping 2013; 34: 1330-1343), whereas others have reported that gender has a relatively small (Bluhm et al., NeuroReport 2008; 19: 887-891) or lack of effect (Weissman-Fogel et al., Human Brain Mapping 2010) on resting state networks. Thus, further studies are needed to clarify the impact of gender and ApoE4 genotype on EEG metrics.

To conclude, as shown by this example the present invention proposed several EEG neuromarkers that are effective in the evaluation of a neurodegeneration index that may be used for discriminating healthy controls subjects from preclinical Alzheimer's disease individuals with a high risk of future cognitive decline. As these EEG neuromarkers are modulated by the degree of severity of amyloid load, the neurodegeneration index helps to distinguish between an early and a late phase of preclinical AD.

### Example 2

Observational Study Design and Participants

This example was based on a cohort including baseline data of 314 cognitively normal individuals, between 70 and 85 years old, with subjective memory complaints and unimpaired cognition [Mini Mental State Examination (MMSE) score 527 and Clinical Dementia Rating score 0], no evidence of episodic memory deficit [Free and Cued Selective Reminding Test (FCSRT) total recall score 541]. Demographic, cognitive, functional, biological, genetic, genomic, imaging including brain structural and functional MRI, ¹⁸F-FDG PET and ¹⁸F-florbetapir PET electrophysiological and other assessments were performed at baseline and regularly during follow-up. EEGs were performed every 12 months.

To evaluate if EEG metrics' changes were a consequence of neurodegeneration, amyloid burden, or a combination of the two, the whole cohort was divided into four groups of subjects depending on their amyloid status (evidenced by ¹⁸F-florbetapir PET) and neurodegeneration status (revealed by ¹⁸F-FDG PET). The first group was amyloid-positive and neurodegeneration-positive (A+N+), which corresponds to stage 2 of preclinical Alzheimer's disease according to Sperling et al. (Toward defining the preclinical stages of Alzheimer's disease: recommendations from the National Institute on Aging-Alzheimer's Association workgroups on diagnostic guidelines for Alzheimer's disease. Alzheimers Dement, 2011). The second group was amyloid-positive and neurodegeneration-negative (A+N−), which corresponds to stage 1 of preclinical Alzheimer's disease according to Sperling et al. (2011). These first two groups belong to Alzheimer's disease continuum according to Jack et al. (NIA-AA research framework: toward a biological definition of Alzheimer's disease. Alzheimers Dement 2018). The third group was amyloid-negative and neurodegeneration-positive (A−N+), which corresponds to ‘suspected non-Alzheimer's pathophysiology’ (SNAP) (Jack et al., An operational approach to National Institute on Aging-Alzheimer's Association criteria for preclinical Alzheimer disease. Ann Neurol 2012; 2012). The last group was the control group, defined by amyloid-negative and neurodegeneration-negative subjects (A−N−).

The subjects into four groups was classified based on amyloid status (evidenced by ¹⁸F-florbetapir PET) and neurodegeneration status (evidenced by ¹⁸F-FDG PET brain metabolism in Alzheimer's disease signature regions): A+N+, A+N−, A−N+ and A−N− (control group).

PET Acquisition and Processing

PET scans were acquired 50 min after injection of 370 MBq (10 mCi) ¹⁸F-florbetapir or 30 min after injection of 2 MBq/kg ¹⁸F-FDG. Reconstructed images were analyzed and a ¹⁸F-florbetapir-PET standardized uptake value ratio (SUVR) threshold of 0.7918 was used to dichotomize subjects into amyloid-positive and -negative groups (Dubois et al., Cognitive and neuroimaging features and brain b-amyloidosis in individuals at risk of Alzheimer's disease (INSIGHT-preAD): a longitudinal observational study. Lancet Neurol 2018, and Habert et al., Evaluation of amyloid status in a cohort of elderly individuals with memory complaints: validation of the method of quantification and determination of positivity thresholds. Ann Nucl Med 2018). The same image assessment pipeline was applied to measure brain glucose metabolism on ¹⁸F-FDG PET scans. Cortical metabolic indices were calculated in four bilateral regions of interest that are specifically affected by Alzheimer's disease (Jacket al., 2012): posterior cingulate cortex, inferior parietal lobule, precuneus, and inferior temporal gyms, and the pons was used as the reference region. In this example, subjects were considered neurodegeneration-positive if the mean ¹⁸F-FDG PET SUVR of the four Alzheimer's disease signature regions was <2.27.

EEG Acquisition and Processing

EEG data were acquired with a high-density 256-channel EGI system (Electrical Geodesics Inc.) with a sampling rate of 250 Hz and a vertex reference. During the recording, patients were instructed to keep awake and relaxed. The total length of the recording was 2 min, during which participants alternated 30-s segments of eyes closed and eyes open conditions. Sixty seconds of eyes closed resting state recording were selected for the analysis. For EEG data processing it was used a pipeline that automates processing of EEG recordings with automated artefact removal and extraction of EEG measures (Sitt et al., Large scale screening of neural signatures of consciousness in patients in a vegetative or minimally conscious state. Brain 2014; and Engemann et al., Robust EEG-based cross-site and cross-protocol classification of states of consciousness. Brain J Neurol, 2018). A band-pass filtering (from 0.5 to 45 Hz) and a notch filter at 50 Hz and 100 Hz were applied. Data were cut into 1-s epochs. Bad channels and bad epochs were rejected.

Calculation and Analysis of EEG Metrics

314 high density 256-channel EEG recordings from the cohort baseline data were analysed. For the calculation of EEG metrics, the values of the first 224 electrodes were analyzed, which were the scalp (non-facial) electrodes. For each recording, a set of measures organized were extracted according to a theory-driven taxonomy (Sitt et al., Large scale screening of neural signatures of consciousness in patients in a vegetative or minimally conscious state. Brain 2014). Power spectral density (PSD), median spectral frequency (MSF) and spectral entropy measure dynamics of brain signal at a single electrode site and are based on spectral frequency content. Algorithmic complexity estimates the complexity of a signal based on its compressibility. It measures dynamics of brain signal at a single electrode site and is based on information theory. wSMI is also an information-theoretic metric and estimates functional connectivity between brain regions. For our main analysis, 10 EEG metrics were calculated: PSD in delta (1-4 Hz), theta (4-8 Hz), alpha (8-12 Hz), beta (12-30 Hz), gamma (30-45 Hz), MSF, spectral entropy, algorithmic complexity, wSMI in theta and alpha band. The EEG metrics were averaged across all epochs (60 s recording). PSD was normalized as described in Sitt et al. (2014). In a supplementary analysis, the results of functional connectivity measured by wSMI were compared to two additional ‘traditional’ functional connectivity metrics, namely phase locking value (PLV) and weighted phase lag index (wPLI).

Statistical Analysis

Statistical analyses were performed using R software, version 3.5.0. It was compared baseline characteristics between the four groups using one-way ANOVA for continuous variables and χ² test for categorical variables. When global test was significant, post hoc Tukey test was performed for continuous variables and pairwise χ² test with Benjamini-Hochberg correction for categorical variables, to determine which groups differed from each other.

First, local regression (LOESS) was used to study the relationship between average EEG metrics (mean value across all scalp electrodes), mean amyloid SUVR and mean ¹⁸F-FDG SUVR.

To study the impact of amyloid load, brain metabolism, age, gender, educational level, APOE ε⁴ and hippocampal volume on EEG metrics, two types of analyses were performed. The first analysis was on the mean value of each metric across all scalp (non-facial) electrodes. The second was on the value of each metric at each scalp electrode so there were 224 values for each metric per participant. For wSMI, connectivity measures were summarized by calculating the median value from each electrode to all the other electrodes. Multiple models were performed to evaluate the impact of main effects and interactions. Type II tests were performed. Pvalues were corrected for multiple testing on 10 measures with the Benjamini-Hochberg false discovery rate (BH-FDR) procedure.

For the analysis of average EEG metrics, multiple linear regressions were performed. Simple linear regressions were first performed to evaluate if amyloid load or brain metabolism should be included as categorical variables (A+, A−, N+, N−) or as continuous variables (amyloid SUVR, mean ¹⁸F-FDG SUVR), by maximizing the coefficient of determination R2, depending on the EEG metrics. The effects of interest were included in multiple models as well as interaction between amyloid load and brain metabolism.

For the analysis of the value of each metric at each electrode, linear mixed models were carried out with the effects of interest as fixed effects as well as the electrode number, and the subject as random effect. Interactions between amyloid load, brain metabolism and electrode number were included in the models as well as all two-way interactions between these three effects. A cluster-based permutation test was performed with a threshold-free cluster enhancement (TFCE) method (Smith and Nichols, 2009) to correct for multiple comparisons on 224 electrodes and to see which electrodes showed statistically significant differences for pairwise comparisons between the following groups: A+N+ versus A−N−, A+N− versus A−N−, A−N+ versus A−N−, A+ versus A−, and N+ versus N−. A scalp topographical maps was generated using MNE-Python (Gramfort et al., MEG and EEG data analysis with MNE-Python. Front Neurosci 2013).

To provide anatomically based interpretation of neural activity, a source level functional connectivity analysis was done on a representative sample of the four groups of participants.

Results

The mean age of all participants was 76.1 years [standard deviation (SD) 3.5] and 67.8% of the participants had a high educational level. There were no differences between the four groups for age and educational level. There were more females in A−N− (66.3%) and A+N− (74.6%) groups compared to A+N+ group (36.0%). The proportion of APOE e4 carriers was higher in A+N+ and A+N− groups than in A−N+ and A−N− groups (44.0% and 34.9% versus 5.9% and 14.3%, respectively). The four groups did not differ for cognitive scores except for the FCSRT delayed free recall where A+N+ group had significantly lower scores than A+N− and A−N− groups [10.4 (SD 2.5) versus 11.8 (SD 2.3) and 12.0 (SD 2.1), respectively]. The mean ¹⁸F-FDG PET SUVR was 2.2 (SD 0.1) in the A+N+ group, 2.2 (SD 0.1) in the A−N+ group, 2.5 (SD 0.2) in the A+N− group and 2.6 (SD 0.2) in the A−N− group. The mean amyloid SUVR was 1.1 (SD 0.2) in the A+N+ group, 1.0 (SD 0.2) in the A+N− group, 0.7 (SD 0.1) in the A−N+ group and 0.7 (SD 0.1) in the A−N− group. The total hippocampal volume measured on structural MRI was significantly lower in A+N+ subjects compared to A−N− subjects [2.6 (SD 0.2) versus 2.8 (SD 0.3), respectively].

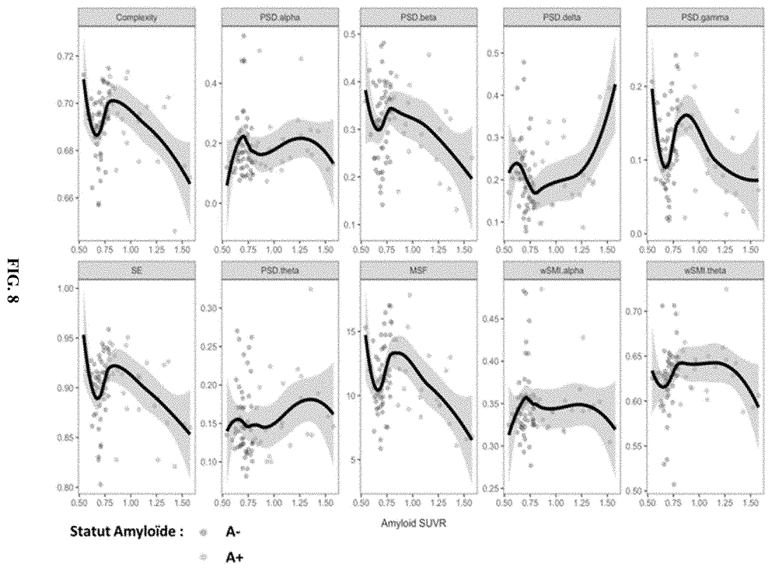

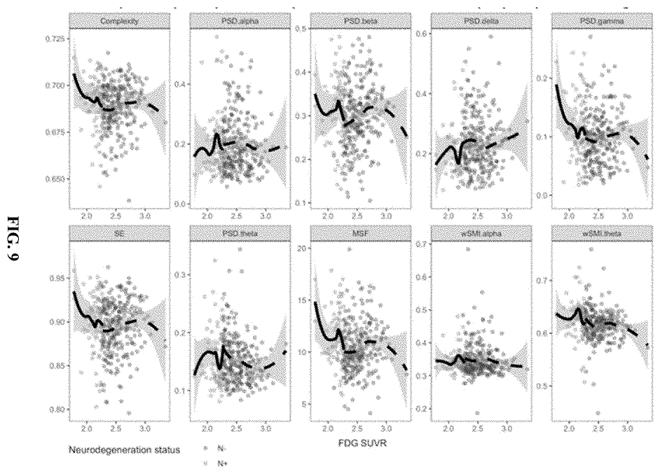

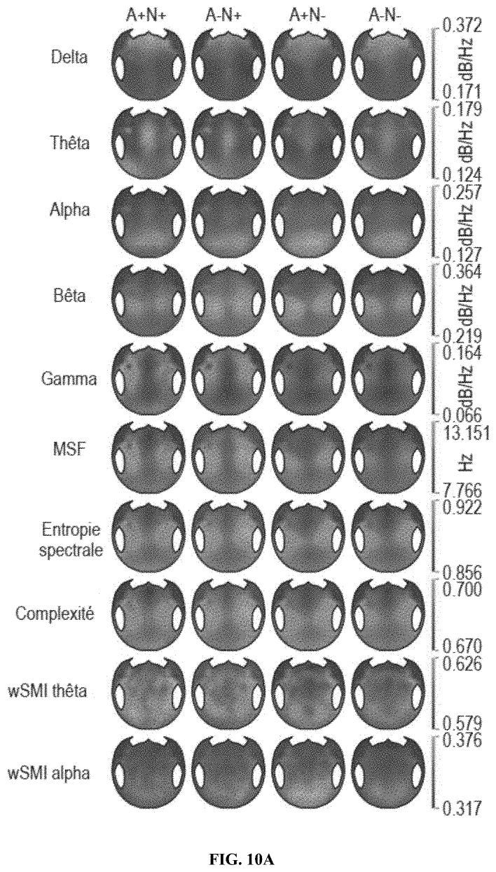

As a first exploratory step, local regression was used to study the relationship between average EEG metrics and mean amyloid SUVR (FIG. 7) and mean ¹⁸F-FDG SUVR (FIG. 9). The relationship between amyloid SUVR and PSD delta followed a U-shape curve whereas the relationship between amyloid SUVR and PSD beta, PSD gamma, MSF, spectral entropy and complexity followed an inverted U-shape curve. Amyloid SUVR inflection points values were between 0.96 and 0.98 for all the previous EEG measures. The relationship was less clear between amyloid burden, PSD alpha and PSD theta. The degree of severity of amyloid load did not seem to have an impact on wSMI theta and wSMI alpha. To better understand the relationship between amyloid load and EEG metrics it was done local regression of average EEG metrics on amyloid SUVR first for N+ subjects only (FIG. 8) and second for N− subjects only. Interestingly, in N+ subjects, local regression of EEG metrics on amyloid SUVR showed much more obvious inverted U-shape curves for intermediate to very high amyloid load than the previous regression on the whole cohort, for PSD beta, PSD gamma, MSF, spectral entropy, complexity and also for wSMI theta. Moreover, in N+ subjects, the relationship between PSD delta and amyloid SUVR followed a more pronounced U-shape curve. After exceeding a certain level of amyloid load, complexity, spectral entropy, MSF, PSD beta, PSD gamma and wSMI theta decreased markedly and PSD delta increased noticeably. Amyloid burden did not show any noticeable effect on EEG measures in N− subjects. To summarize, the degree of severity of amyloid burden had a strong impact on EEG metrics in the presence of neurodegeneration, with increased high frequency oscillations for intermediate amyloid burden and a slowing of brain oscillations for high to very high amyloid load.

Local regression of average EEG metrics on mean ¹⁸F-FDG SUVR (FIG. 9) showed a trend towards increased complexity, PSD beta, PSD gamma, spectral entropy, MSF and wSMI theta and decreased PSD delta when brain metabolism decreased. The relations between brain metabolism, PSD alpha and PSD theta were less clear. The level of brain metabolism did not seem to have an impact on wSMI alpha. Similar trends were found in local regression of EEG metrics on ¹⁸F-FDG SUVR separately for A+ and A− subjects. Thus, as a main effect, neurodegeneration in Alzheimer's disease signature regions seemed to increase high frequency oscillations, complexity, spectral entropy and functional connectivity measured by wSMI theta, except when neurodegeneration was associated with very high amyloid load, where the trend of EEG metrics reversed.

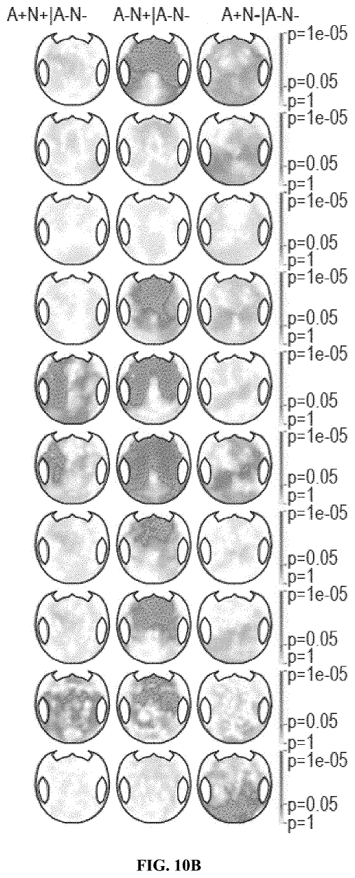

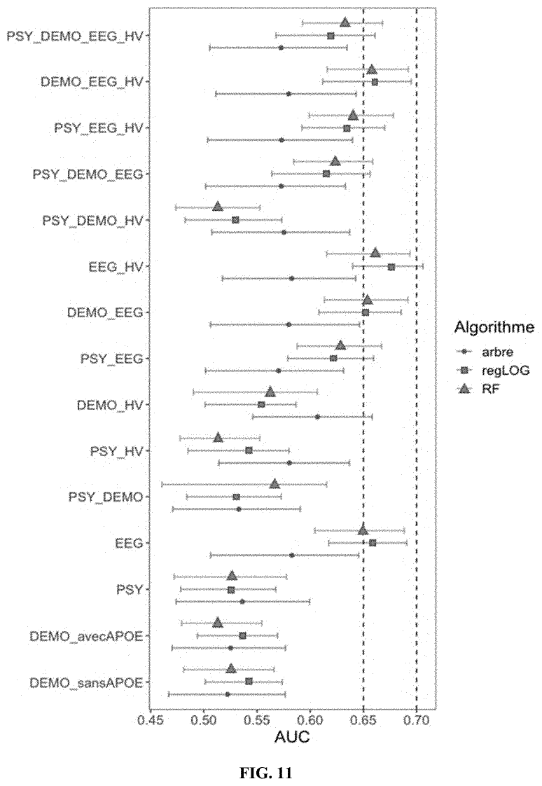

Topographical differences were evaluated across EEG measures between the control group (A−N−) and the three other groups (A+N+, A+N− and A−N+) (FIG. 10A-10B). The objectives were to assess the discrimination capacity of the different EEG metrics between groups and to better understand the impact of amyloid and neurodegeneration on EEG measures. All P-values were adjusted on APOE ε⁴ status, gender, education level, age and hippocampal volume. The A−N+ group showed maximum EEG changes compared to A−N− control group. A−N+ subjects had lower PSD delta in frontocentral regions and right temporal region, higher PSD beta, complexity, spectral entropy and wSMI theta in frontocentral regions and higher PSD gamma in frontocentral and temporal bilateral regions, compared to A−N− group. The A−N+ group presented a widespread increase of MSF in frontocentral and parietotemporal regions. Thus, several EEG measures were efficient indices in discriminating A−N+ subjects from A−N− subjects. The A+N+ group showed only an increase in PSD gamma in left frontotemporal region and a discrete increase in MSF in left temporal region, compared to the A−N− group. The A+N+ group showed a trend towards increased wSMI theta in centro-parieto-temporal regions but did not reach statistical significance. The A+N− group showed significantly increased wSMI alpha in parieto-occipital regions compared to the A−N− group.

Conclusions

It was found a local increase of functional connectivity measured by wSMI alpha in parieto-occipital regions in subjects at stage 1 of preclinical Alzheimer's disease. This could be explained by abnormal transient neuronal hyperexcitability related to amyloid-β deposition with a relative decrease in synaptic inhibition. The ‘acceleration’ hypothesis suggests that once amyloid-β deposition is initiated by independent events, a milieu of higher functional connectivity hastens this deposition, which eventually leads to the functional disconnection or metabolic deterioration in the subjects with amyloid burden. The metabolic demands associated with high connectivity may be the detrimental phenomenon that triggers downstream cellular and molecular events associated with Alzheimer's disease. Previous work in animal models has shown that intermediate levels of amyloid-β enhance synaptic activity presynaptically, whereas abnormally high levels of amyloid-β impair synaptic activity by inducing post-synaptic depression. This is consistent with our results showing basically two different EEG phases in preclinical Alzheimer's disease stage 2. In the early preclinical stage that is characterized by neurodegeneration combined with intermediate levels of amyloid-β, there is an increase in brain oscillations and functional connectivity due to compensation and/or amyloid-β-related excitotoxicity. Then, the increase in brain oscillations and functional connectivity would hasten amyloid-β deposition. In a later preclinical stage characterized by neurodegeneration combined with high to very high levels of amyloid-β, there is a slowing of brain oscillations and reduced functional connectivity due to compensatory mechanisms failure and/or post-synaptic depression, with an EEG pattern getting close to the one observed in MCI and Alzheimer's disease. The breakdown of initial functional compensation would facilitate accelerated tau-related neurodegenerative processes

In this example, it is showed that a decrease in brain metabolism in Alzheimer's disease signature regions was associated with higher theta power.

To conclude, this second example performed on a wider population compared to the first example, shows that several EEG neuromarkers that are effective in the evaluation of a neurodegeneration index that may be used for identifying individuals at high risk of preclinical Alzheimer's disease and future cognitive decline. Moreover, EEG biomarkers seem to be useful tools to measure and monitor neurodegeneration. As these EEG neuromarkers are modulated by the degree of severity of amyloid load, the neurodegeneration index helps to distinguish between an early and a late phase of preclinical AD.

### Example 3

In this example, machine learning analysis was used to evaluate, at the individual level, the performance of EEG biomarkers to identify amyloid status (A+ versus A−) and neurodegeneration status (N+ versus N−).

The EEG is particularly interesting among the different measures available to distinguish N+ participants from N− participants at the individual level (FIG. 11).

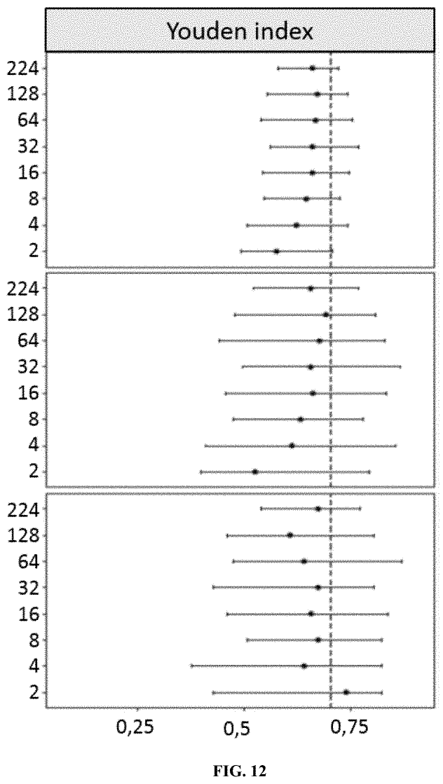

The reduction in the number of electrodes only affects diagnostic performance when only 2 electrodes are used (FIG. 12) and then the sensitivity remains good at 74%. At the expense of specificity. The set of 4 electrodes (2 frontal and 2 parietal) gives good results to diagnose Alzheimer's neurodegeneration in this preclinical phase with a sensitivity at 64% and a specificity at 61%.

This example also show that the most strongly predictive parameters of amyloid status were first the ApoE4 genotype, then demographic parameters with age, sex, education level, and to a much lesser degree the hippocampal volume measured in MRI.

1-16. (canceled)

17. A system to measure and monitor neurodegeneration of a subject, comprising at least one processor configured to:
acquire electroencephalographic signals with multiple EEG channels from a subject perceptually isolated;
extract at least one EEG metric representative of neurodegeneration;
evaluate the at least one EEG metric and extract a neurodegeneration index based on the evaluation of the at least one EEG metrics; and
at least one output configured to provide the neurodegeneration index.

18. The system according to claim 17, wherein the at least one processor is configured to extract at least one EEG metric selected from the group of: weighted symbolic mutual information in at least one frequency band, power spectral density calculated in at least one frequency band, median spectral frequency, spectral entropy and algorithmic complexity.

19. The system according to claims 17, wherein in order to extract the weighted symbolic mutual information, the at least one processor is configured to perform a symbolic transformation of the electroencephalographic signals into a series of discreate symbols and calculating the weighted symbolic mutual information using said series of discrete symbols.

20. The system according to claim 18, wherein the weighted symbolic mutual information is calculated in the theta frequency band.

21. The system according to claim 17, wherein the multiple EEG channels comprises at least two EEG channels.

22. The system according to claim 17, wherein the neurodegeneration index is representative of the neurodegeneration affecting a subject suffering from preclinical Alzheimer's disease.

23. The system according to claim 22, wherein the neurodegeneration index is representative of a stage of preclinical Alzheimer's disease affecting the subject.

24. The system according to claim 18, wherein the power spectral density is calculated in the delta frequency band, theta frequency band, alpha frequency band, beta frequency band and/or in the gamma frequency band.

25. The system according to claim 17, wherein the EEG metrics further comprises at least one of the following median spectral frequency, spectral entropy or algorithmic complexity.

26. The system according to claim 17, wherein the at least one processor is configured to extract the neurodegeneration index from the comparison of the at least one EEG metrics with at least one predefined threshold.

27. The system according to claim 17, wherein the at least one processor is further configured to pre-process the electroencephalographic signals.

28. A computer-implemented method for measuring and monitoring neurodegeneration of a subject, comprising the steps of:
receiving electroencephalographic signals acquired with multiple EEG channels from a subject perceptually isolated;
extracting at least one EEG metric representative of neurodegeneration;
evaluating the at least one EEG metric and extracting a neurodegeneration index; and
outputting the neurodegeneration index.

29. The computer-implemented method according to claim 28, wherein the at least one EEG metric extracted is selected from the group of: weighted symbolic mutual information in at least one frequency band, power spectral density calculated in at least one frequency band, median spectral frequency, spectral entropy and algorithmic complexity.

30. The computer-implemented method according to claim 28, further comprising performing a symbolic transformation of the electroencephalographic signals into a series of discreate symbols and calculating the weighted symbolic mutual information using said series of discrete symbols so as to extract the weighted symbolic mutual information.

31. The computer-implemented method according to claim 29, wherein the weighted symbolic mutual information is calculated in the theta frequency band.

32. The computer-implemented method according to claim 29, the power spectral density is calculated in the delta frequency band, theta frequency band, alpha frequency band, beta frequency band and/or in the gamma frequency band.

33. The computer-implemented method according to claim 29, wherein the EEG metrics further comprises at least one of the following median spectral frequency, spectral entropy or algorithmic complexity.

34. A non-transitory computer-readable storage medium comprising instructions that when executed by a computer, causes the computer to carry out the steps of the method according to claims 28.
